SEQUENCE LISTING (6 sequences)

SEQ ID NO: 1 - DNA, 10 bp, Artificial
  Synthetic Single Strand DNA
tacagtgact

SEQ ID NO: 2 - DNA, 10 bp, Artificial
  Synthetic Single Strand DNA
tacagtgact

SEQ ID NO: 3 - DNA, 30 bp, Artificial
  Synthetic Single Strand DNA
tttctctagacaattgtgcaatacgtcttt

SEQ ID NO: 4 - DNA, 50 bp, Artificial
  Synthetic Single Strand DNA
tttctgttgacaatttatcatcggtcgtataatgtgtggaattggtcttt

SEQ ID NO: 5 - DNA, 75 bp, Artificial
  Synthetic Single Strand DNA
tttctggttaaggtgtgtatatgctcggctactaattagtgagtattattctcgctactattaacagttgtcttt

SEQ ID NO: 6 - DNA, 100 bp, Artificial
  Synthetic Single Strand DNA
tttctggttaaggtgtgtaatatgctcagctactaattaacagttgtctaagctggttaacgtgagtaatatgatcagctactatttaacagttgtcttt

(19) United States
(12) Patent Application Publication
(10) Pub. No.: US 2006/0178507 A1
(43) Pub. Date: Aug. 10, 2006

(54) Fluorous oligonucleotide reagents and affinity purification of oligonucleotides

(75) Inventors: David A. Berry, Ann Arbor, MI (US); William H. Pearson, Ann Arbor, MI (US)

Correspondence Address: YOUNG & BASILE, P.C., 3001 WEST BIG BEAVER ROAD, SUITE 624, TROY, MI, 48084, US

(73) Assignee: BERRY & ASSOCIATES, INC.

(21) Appl. No.: 11/320,218

(22) Filed: Dec. 28, 2005

Related U.S. Application Data
(60) Provisional application No. 60/640,871, filed on Dec. 30, 2004.

(51) Int. Cl. C07H 21/04

(52) U.S. Cl. 536/25.3; 536/25.4

(57) ABSTRACT

Fluorous-tagged oligonucleotide reagents and an oligonucleotide purification methodology making use thereof, the method comprising: Synthesizing oligonucleotides using oligonucleotide reagents each bearing at least one fluorous group to yield a mixture of synthesis products and reagents, the mixture including at least one target synthesized oligonucleotide bearing at least one fluorous group; passing the mixture through a separation medium having an affinity for the at least one fluorous group so that the target synthesized oligonucleotide bearing at least one fluorous group is adsorbed by the separation medium; washing the separation medium with at least a first solvent to selectively dissociate therefrom substantially all synthesis products and reagents of the heterogenous mixture other than the at least one target synthesized oligonucleotide bearing at least one fluorous group; and subsequently dissociating the at least one synthesized oligonucleotide from the separation medium, with or without the fluorous group.

### CROSS-REFERENCE TO RELATED APPLICATIONS

This application is related to, and claims the benefit of priority from, U.S. Provisional Patent Application Ser. No. 60/640,871, filed Dec. 30, 2004.

### STATEMENT REGARDING FEDERALLY SPONSORED RESEARCH OR DEVELOPMENT

This invention was made with government support under SBIR Grant No. R43GM071153. The Government has certain rights in this invention.

### INCORPORATION BY REFERENCE OF MATERIAL SUBMITTED ON A COMPACT DISC

Not applicable.

### FIELD OF THE INVENTION

The present invention pertains to reagents for incorporation with oligonucleotides, the reagents comprising one or more permanent or temporarily associated fluorous groups, as well as a methodology for purifying oligonucleotides synthesized with one or more such reagents.

### BACKGROUND

The automated synthesis of oligonucleotides has fueled the biotechnology revolution, with synthetic oligonucleotides finding application in DNA sequencing, PCR amplification, gene therapy, site-specific mutagenesis, gene cloning, hybridization, etc.

Oligonucleotides are most commonly prepared using automated solid-phase chemistry featuring Köster's 2-cyanoethyl modification of Carruthers' phosphoramidite coupling technique, whether on microgram or kilogram scale. While there are many variations, especially when synthesizing modified oligonucleotides, description of a common oligonucleotide synthetic pathway can be found in Current Protocols in Nucleic Acid Chemistry, Beaucage, S. L.; Bergstrom, D. E.; Glick, G. D.; Jones, R. A., Eds., John Wiley & Sons, Inc.: New York, Chapters 1-4, 2000-2004. By way of summary (such techniques being well-known to those skilled in the art), synthesis generally comprises anchoring a nucleoside bearing an acid-labile 5′-O-(4,4′-dimethoxytrityl) (“DMTr”) group to controlled-pore glass via a tether to its 3′-hydroxyl group. Assembly of the desired oligonucleotide is then carried out by repeating four basic steps: (1) Deblocking of the 5′-DMTr group with acid; (2) coupling of the resultant free 5′-hydroxyl group with a 5′-O-DMTr-3′-O-(2-cyanoethyl-N,N-diisopropylphosphoramidyl) nucleoside (a “phosphoramidite”) in the presence of an activator such as tetrazole; (3) capping of unreacted 5′-hydroxyl groups by acylation (e.g., with acetic anhydride); and (4) oxidation of the resultant phosphite to the phosphate oxidation level. After the installation of the appropriate number and type of monomers, treatment with ammonia or related nucleophiles serves to cleave the chain from the solid support and de-protect the nucleobase amino groups. Detritylation of the final 5′-O-DMTr group with acid can be performed before or after ammonia treatment to afford the final oligonucleotide.

A long-standing problem in oligonucleotide synthesis, and a particularly relevant issue in the manufacture of therapeutic oligonucleotides, is achieving product purity. While automated synthesis is the best available strategy for the production of oligonucleotides, heterogeneous mixtures of products are generated, complicating purification and limiting scale-up. And though the solid-phase synthesis method allows convenient introduction and removal of large excesses of chemical reagents, the reactions still do not proceed to completion. Every chemical reaction in the synthesis (detritylation, coupling, capping, oxidation, ammonolysis) proceeds in less than 100% yield and produces impurities. Notably, each phosphoramidite coupling step leaves a small amount of truncated material as a result of incomplete coupling. If these materials were allowed to react in the next coupling cycle, unwanted deletion mutants would result. This problem is addressed to a large extent by capping the unreacted 5′-hydroxyl groups with an acylating agent such as acetic anhydride. These capped products end up as shorter oligonucleotides (so-called “failure sequences”) after the final cleavage and deprotection chemistry is carried out at the end of the synthesis. Unfortunately, the capping step is not quantitative, leaving uncapped 5′-hydroxyls that react in the next phosphoramidite coupling, which ultimately produces near full-length molecules (“deletion sequences”) that contain internal deletions, i.e. n-1-mer, n-2-mer, etc.

The foregoing problems are further exacerbated as the length of the synthesized oligonucleotide strand increases. For example, a 20-mer (>100 chemical steps) is typically produced in 40-70% yield, depending on scale, the remaining 30-60% being a heterogeneous mixture of undesired oligonucleotides (including failure and deletion sequences). A crude 40-mer contains about 65% of undesired material. Longer oligonucleotides (75-mer and up) are produced in low yield and low purity, limiting the utility of these potentially important materials. And above 100 nucleotides, the limits of solid-phase synthesis are soon reached as the overall yield diminishes to an unusable level while the purity continues to drop.

While some applications (e.g., sequencing or PCR amplification) do not require highly pure oligonucleotides, many others, including, for example, mutagenesis, Q-PCR, end labeling, kinasing, gel shift assays, gene construction, therapeutics, and cloning/expression applications, as well as applications requiring modified oligonucleotides (e.g. diagnostic probes bearing fluorophores, biotins, etc.), necessitate high quality materials, so the researcher must painstakingly purify these materials using a combination of separation techniques, then analyze and quantify these materials, resulting in losses in time, money, and substantial quantities of the oligonucleotide itself.

Several methods have heretofore been developed to purify oligonucleotides, including anion-exchange (AX) chromatography, reverse phase (RP) chromatography, polyacrylamide gel electrophoresis (PAGE), and affinity chromatography. These methods may be employed individually or in combination to achieve even higher levels of purity. However, each method has its limitations. PAGE is time-consuming, is limited to the purification of small quantities, and affords low material recoveries. AX- and RP-HPLC are also time-consuming, require expensive columns and instruments, often require tedious process development, and can show insufficient resolution between the desired and undesired oligonucleotides, especially with longer sequences. Solid-phase extraction (SPE) techniques based on RP cartridges and tubes can significantly speed up the purification process, but current SPE methods are limited to relatively short oligonucleotides and often show low recoveries. And affinity methods, while showing promise, often require tedious and expensive methodology.

Accordingly, there continues to exist the need for a simple, economical, and effective means for purifying oligonucleotides.

### SUMMARY OF THE DISCLOSURE

The specification addresses the foregoing needs and disadvantages attending conventional oligonucleotide purification techniques, and encompasses other features and advantages, in the disclosure of both oligonucleotide reagents oligonucleotide synthesis, the oligonucleotide reagents each bearing, either permanently or temporarily (i.e., via a removable protecting group), at least one fluorous group, as well as a methodology for the purification of oligonucleotides synthesized from one or more such reagents which takes advantage of the heightened affinity between the at least one fluorous group and the separation media.

In one aspect thereof, the present invention comprehends a method for the purification of such fluorous “tagged” oligonucleotides comprising the steps of:

(a) Synthesizing at least one oligonucleotide using at least one oligonucleotide reagent bearing at least one fluorous group to yield a heterogenous mixture of oligonucleotide synthesis products and reagents, said mixture including at least one target synthesized oligonucleotide bearing at least one fluorous group;

(b) passing said mixture through a separation medium having an affinity for the at least one fluorous group so that the at least one target synthesized oligonucleotide bearing at least one fluorous group is adsorbed by said separation medium;

(c) washing the separation medium with at least a first solvent to selectively dissociate therefrom substantially all synthesis products and reagents of the heterogenous mixture other than the at least one target synthesized oligonucleotide bearing at least one fluorous group; and

(d) subsequently dissociating the at least one target synthesized oligonucleotide from the separation medium, with or without the at least one fluorous group.

In one embodiment of the foregoing methodology, the at least one oligonucleotide reagent comprises a protected nucleoside the protecting group of which bears the at least one fluorous group, the at least one target synthesized oligonucleotide comprises the protected nucleoside, and wherein further the step (d) comprises removing from the at least one target synthesized oligonucleotide the protecting group bearing the at least one fluorous group, and thereafter eluting said at least one target synthesized oligonucleotide from the separation medium without the protecting group bearing the at least one fluorous group. In one form of this embodiment, the at least one target synthesized oligonucleotide bearing at least one fluorous group comprises, at the 5′ terminus thereof, a single protected nucleoside the protecting group of which bears at least one fluorous group.

In a second embodiment of the inventive method, the step (d) comprises washing the separation medium with at least a second solvent more fluorophilic than said at least first solvent to dissociate from said separation medium the at least one target synthesized oligonucleotide bearing at least one fluorous group. According to one form thereof, the at least one oligonucleotide reagent comprises a protected nucleoside the protecting group of which bears the at least one fluorous group, the at least one target synthesized oligonucleotide comprises the protected nucleoside, and wherein the method comprises the further ordered step (e) of removing from the at least one target synthesized oligonucleotide the protecting group bearing the at least one fluorous group. The at least one target synthesized oligonucleotide bearing at least one fluorous group may comprise, at the 5′ terminus thereof, a single protected nucleoside the protecting group of which bears at least one fluorous group.

According to either embodiment of the inventive method, the separation medium comprises fluorous affinity groups.

Per one feature hereof, the separation medium comprises a reverse-phase adsorbent bearing fluorinated groups.

According to another feature, the separation medium comprises a polymeric matrix bearing fluorinated oligonucleotide groups. The polymeric matrix may, per another feature hereof, be chosen from poly(divinylbenzene) or polystyrene cross-linked with divinylbenzene.

Per yet another feature of this invention, the separation medium comprises a silica matrix bearing fluorinated groups.

According to still another feature, the separation medium may be a lipophilic reverse-phase adsorbent based on a matrix of silica, poly(divinylbenzene) or polystyrene cross-linked with divinylbenzene.

The present invention further encompasses various oligonucleotide reagents for oligonucleotide synthesis, these reagents all most generally characterized in bearing at least one fluorous group, either permanently or via an otherwise conventional protecting group such as, for instance, DMTr, Boc, TIPS, TES, etc.

In one embodiment hereof, such oligonucleotide reagents comprise at least one fluorous protecting group, and are characterized by the following nominal formula (I): 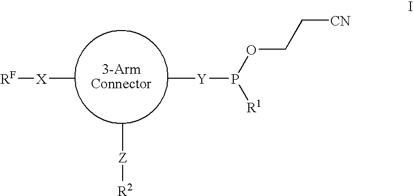
Wherein, X is selected from the group consisting of O, N, and S; Y is O or S; Z is absent, or is selected from the group consisting of O, N, and S; R¹ is selected from the group consisting of N(CH₃)₂, N(C₂H₅)₂, N(C₃H₇)₂, N(CH(CH₃)₂)₂, 1-pyrrolidinyl, 1-piperidinyl, 4-morpholinyl, and 1-imidazolyl; R² is selected from the group consisting of a natural nucleobase, an unnatural nucleobase, a fluorescent tag, a quencher tag, biotin, and a solid phase synthesis support; R^{F} is a fluorous protecting group selected from the group consisting of {CₙF₂ₙ₊₁—(CH₂)ₘ}DMTr, {CₙF₂ₙ₊₁—(CH₂)ₘ}MMTr, {CₙF₂ₙ₊₁—(CH₂)ₘ}Tr, {CₙF₂ₙ₊₁—(CH₂)ₘ}(Ph)₂CH, {CₙF₂ₙ₊₁—(CH₂)ₘ}PhCH₂, {CₙF₂ₙ₊₁—(CH₂)ₘ}TBDMS, {CₙF₂ₙ₊₁—(CH₂)ₘ}TES, {CₙF₂ₙ₊₁—(CH₂)ₘ}TIPS, {CₙF₂ₙ₊₁—(CH₂)ₘ}Boc, and {CₙF₂ₙ₊₁—(CH₂)ₘ}Cbz, wherein n is 4-12, m is 1-4, and R is a straight or branched alkyl of 1-4 carbon atoms; and the three-arm connector is selected from the group consisting of: 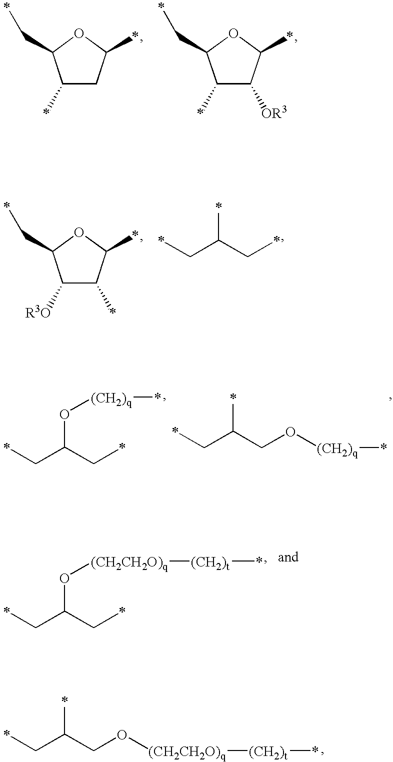
wherein * represents attachment points for X, Y and Z, q is 2-12, t is 2-4, and R³ is selected from the group consisting of CH₃CO, (CH₃)₂CHCO, (CH₃)₂CHCH₂CO, (CH₃)₃CCO, PhCO, (CH₃)₃CSi(CH₃)₂, and (C₂H₅)₃Si.

Exemplary compounds according to this embodiment which are described herein include natural (i.e., DNA and RNA) phosphoramidites, unnatural nucleoside phosphoramidites, fluorescent tags, quencher tags, and biotin tags.

In a second embodiment, the oligonucleotide reagents of the present invention comprise at least one fluorous protecting group, and are characterized by the following nominal formula (II): 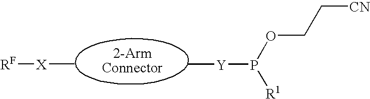
Wherein, X is selected from the group consisting of O, N, and S; Y is O or S; R¹ is selected from the group consisting of N(CH₃)₂, N(C₂H₅)₂, N(C₃H₇)₂, N(CH(CH₃)₂)₂, 1-pyrrolidinyl, 1-piperidinyl, 4-morpholinyl, and 1-imidazolyl; R^{F} is selected from the group consisting of {CₙF₂ₙ₊₁—(CH₂)ₘ}DMTr, {CₙF₂ₙ₊₁—(CH₂)ₘ}MMTr, {CₙF₂ₙ₊₁—(CH₂)ₘ}Tr, {CₙF₂ₙ₊₁—(CH₂)ₘ}(Ph)₂CH, {CₙF₂ₙ₊₁—(CH₂)ₘ}PhCH₂, {CₙF₂ₙ₊₁—(CH₂)ₘ}TBDMS, {CₙF₂ₙ₊₁—(CH₂)ₘ}TES, {CₙF₂ₙ₊₁—(CH₂)ₘ}TIPS, {CₙF₂ₙ₊₁—(CH₂)ₘ}Boc, and {CₙF₂ₙ₊₁—(CH₂)ₘ}Cbz, wherein n is 4-12, m is 1-4, and R is straight or branched alkyl of 1-4 carbon atoms; and the two-arm connector is selected from the group consisting of *—(CH₂)_{q}—*, *—(CH₂CH₂O)_{q}—(CH₂)ₜ—*, *—(CH₂CH₂CH₂O)_{q}—(CH₂)ₜ—*, 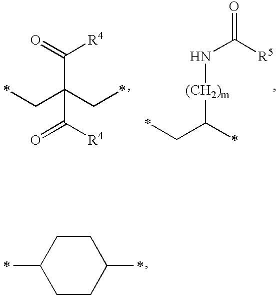
and *—(CH₂)_{q}—S—S—(CH₂)_{q}—*, and in which group * signifies attachment points for X and Y, q is 2-12, t is 2-4, m is 1-4, R⁴ is OCH₃ or NH₂, and R⁵ is selected from the group consisting of H, CF₃, CH₃, OC(CH₃)₃, and OCH₂Ph.

Exemplary reagents according to this first alternative embodiment which are described herein include amino modifiers, thiol modifiers, universal fluorous phosphoramidites, and permanent fluorous tags.

In a third embodiment, oligonucleotide reagents of the present invention comprise at least one fluorous protecting group, and are characterized by the following nominal formula (III): 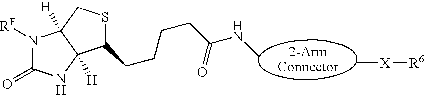
Wherein, X is selected from the group consisting of O, N and S; R⁶ is selected from the group consisting of H, ICH₂CO—*, 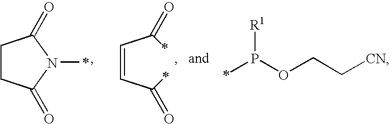
wherein R¹ is N(CH₃)₂, N(C₂H₅)₂, N(C₃H₇)₂, N(CH(CH₃)₂)₂, 1-pyrrolidinyl, 1-piperidinyl, 4-morpholinyl, and 1-imidazolyl; R^{F} is selected from the group consisting of {CₙF₂ₙ₊₁—(CH₂)ₘ}DMTr, {CₙF₂ₙ₊₁—(CH₂)ₘ}MMTr, {CₙF₂ₙ₊₁—(CH₂)ₘ}Tr, {CₙF₂ₙ₊₁—(CH₂)ₘ}(Ph)₂CH, {CₙF₂ₙ₊₁—(CH₂)ₘ}PhCH₂, {CₙF₂ₙ₊₁—(CH₂)ₘ}Boc, and {CₙF₂ₙ₊₁—(CH₂)ₘ}Cbz, wherein n is 4-12, and m is 1-4; and the two-arm connector is selected from the group consisting of *—(CH₂)_{q}—*, *—(CH₂)_{q}CO—* *—(CH₂CH₂O)_{q}—(CH₂)ₜ—*, *—(CH₂CH₂CH₂O)_{q}—*, 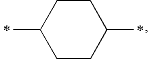
and *—(CH₂)_{q}—S—S—(CH₂)_{q}—*, and in which group * signifies attachment points for X and NH, q is 2-12, t is 2-4, R⁴ is OCH₃ or NH₂, and R⁵ is selected from the group consisting of H, CF₃, CH₃, OC(CH₃)₃, and OCH₂Ph.

Exemplary reagents according to this embodiment which are described herein include biotin tags.

In a fourth embodiment, the oligonucleotide reagents of the present invention comprise at least one permanently incorporated fluorous group, and are characterized by the following nominal formula (IV): 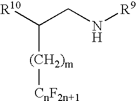
Wherein, n is an integer from 4-12; m is an integer from 1-4; R⁹ is selected from the group consisting of H, Boc, Cbz, COCH₂CH₂CO2H, a fluorescent tag, a quencher tag, biotin, and a solid phase synthesis support; and R¹⁰ is selected from the group consisting of CO₂H, CO₂CH₃, CO₂—(N-succinimidyl), CONH(CH₂)_{q}N-maleimide, CONH(CH₂)_{q}NHCOCH₂I, CONH(CH₂)_{q}NHCOCH₂Br, CONH(CH₂)_{q}OCH₂CH(OR⁸)CH₂OR⁷, CH₂OH, CH₂OP(R¹)OCH₂CH₂CN, CH₂OCH₂CH(OR⁸)CH₂OR⁷, CH₂OCH(CH₂OR⁷) CH₂OR⁸, CH₂O(CH₂)_{q}OR⁷, CH₂O(CH₂CH₂O)_{q}R⁷, and CH₂O(CH₂)_{q}—S—S—(CH₂)_{q}OR⁷, and in which group q is 2-12 R⁷ is one of H, COCH₂CH₂CO2H, DMTr, MMTr, a solid phase synthesis support, and P(R¹)OCH₂CH₂CN, R¹ is one of N(CH₃)₂, N(C₂H₅)₂, N(C₃H₇)₂, N(CH(CH₃)₂)₂, 1-pyrrolidinyl, 1-piperidinyl, 4-morpholinyl, and 1-imidazolyl, and R₈ is one of H, COCH₂CH₂CO2H, DMTr, MMTr, a solid phase synthesis support, and P(R¹)OCH₂CH₂CN, and when R⁷ and R⁸ are both present they are not identical.

Exemplary reagents according to this alternate embodiment which are described herein include fluorescent tags and quencher tags.

In a fifth embodiment of the present invention, the inventive oligonucleotide reagents comprise at least one permanently incorporated fluorous group, and are characterized by the following nominal formula (V): 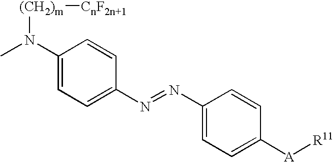
Wherein, m is an integer from 1-4; n is an integer from 4-12; A is CO or SO₂; and R¹¹ is selected from the group consisting of Cl, OH, OCH₃, O—(N-succinimidyl), NH(CH₂)ₜOCH₂CH(OR⁸)CH₂OR⁷, NH(CH₂)_{q}OR⁷, NH(CH₂)ₜO(CH₂CH₂O)_{q}R⁷, and NH(CH₂)_{q}—S—S—(CH₂)_{q}OR⁷, and in which group R⁷ is one of H, COCH₂CH₂CO2H, DMTr, MMTr, a solid phase synthesis support, and P(R¹)OCH₂CH₂CN, R¹ is one of N(CH₃)₂, N(C₂H₅)₂, N(C₃H₇)₂, N(CH(CH₃)₂)₂, 1-pyrrolidinyl, 1-piperidinyl, 4-morpholinyl, and 1-imidazolyl, and R₈ is one of H, COCH₂CH₂CO2H, DMTr, MMTr, a solid phase synthesis support, and P(R¹)OCH₂CH₂CN, and when R⁷ and R⁸ are both present they are not identical.

Exemplary reagents according to this fifth alternate embodiment which are described herein include quencher tags.

### BRIEF DESCRIPTION OF THE DRAWINGS

The foregoing and other features of the instant invention will be better understood with reference to the following description and accompanying drawings, of which:

FIG. 1 depicts a first nominal formula for fluorous-tagged oligonucleotide reagents according to the present invention;

FIGS. 1a through 1f illustrate exemplary fluorous-tagged, protected forms of conventional reagents for oligonucleotide synthesis, according to the nominal formula of FIG. 1;

FIGS. 2a through 2c depict the synthesis of exemplary fluorous-tagged reagents for oligonucleotide synthesis;

FIG. 3 depicts a second nominal formula for fluorous-tagged oligonucleotide reagents according to the present invention;

FIGS. 3a through 3d illustrate exemplary fluorous-tagged, protected forms of conventional reagents for oligonucleotide synthesis and modification, according to the nominal formula of FIG. 3;

FIG. 4 depicts a third nominal formula for fluorous-tagged oligonucleotide reagents according to the present invention;

FIG. 4a illustrates exemplary fluorous-tagged, protected forms of conventional reagents for oligonucleotide synthesis and modification, according to the nominal formula of FIG. 4;

FIG. 5 depicts a fourth nominal formula for fluorous-tagged oligonucleotide reagents according to the present invention;

FIGS. 5a through 5b illustrate exemplary fluorous-tagged forms of conventional reagents for oligonucleotide synthesis and modification, according to the nominal formula of FIG. 5;

FIG. 6 depicts the derivation of exemplary oligonucleotide reagents bearing permanent flourous tags, according to the nominal formula of FIG. 5;

FIG. 7 depicts derivation of further exemplary oligonucleotide reagents bearing permanent flourous tags, according to the nominal formula of FIG. 5;

FIG. 8 depicts a fifth nominal formula for fluorous-tagged oligonucleotide reagents according to the present invention;

FIG. 8a illustrates exemplary fluorous-tagged forms of conventional reagents for oligonucleotide synthesis and modification, according to the nominal formula of FIG. 8;

FIG. 9 is a schematic depicting the fluorous affinity purification method of the present invention;

FIG. 10 depicts exemplary fluorous-tagged oligodeoxyribonucleotides as may be employed in the methodology of the present invention;

FIG. 11 is an HPLC chromatogram of a fluorous-tagged (“F¹”) DMTr-30-mer 19 of FIG. 10 mixed with conventional DMTr-on-30-mer and DMTr-off-30-mer on a FLUOROFLASH 4.6×150 mm HPLC column with mobile phase A=0.1 M TEAA and mobile phase B=MeCN;

FIG. 12 depicts the results of HPLC analysis during SPE purification of a 100-mer oligonucleotide, wherein Trace (a)=the crude synthesis products before purification, Trace. (b)=the eluate from loading of F¹DMTr-100-mer 22 from FIG. 10 onto a FLURO-PAK column, Trace (c)=the eluate from washing the column with 10% acetonitrile in 0.1 M TEAA, and Trace (d)=elution of the DMTr-off 100-mer after on-column detritylation with trifluoroacetic acid;

FIG. 13 is an HPLC chromatogram of a 100-mer derived from purification of the F¹DMTr-100-mer 22 from FIG. 10 with on-column detritylation; and

FIG. 14 is an HPLC chromatogram of a 75-mer derived from purification of the F¹DMTr-100-mer 21 from FIG. 10 with on-column detritylation.

### WRITTEN DESCRIPTION

The following definitions are applicable in this written specification:

“Oligonucleotide” as employed herein means and refers broadly to single-stranded polynucleotides of any length, and is intended by the inventor hereof to comprehend both the DNA (oligodeoxyribonucleotides) and RNA (oligoribonucleotides) forms.

“Oligonucleotide reagent” refers to any compound employed in oligonucleotide synthesis, whether the entire compound only a portion thereof is ultimately incorporated into a synthetic oligonucleotide. Without limitation, exemplary oligonucleotide reagents include nucleoside phosphoramidites employed to incorporate nucleosides into oligonucleotides, spacers, biotins, phosphates, fluorophores, quenchers of fluorescence, amine- and thiol-modifiers, as well as the protected forms (i.e., comprising a protecting group) of such reagents. The related term “oligonucleotide synthesis” is intended to comprehend the employment of oligonucleotide reagents in any act of oligonucleotide creation, including, without limitation, fabrication of synthetic oligonucleotides, as well as the post-fabrication modification thereof.

“Fluorous group” means and refers to a perfluoroalkyl group, linear or branched, attached to a non-fluorous oligonucleotide reagent in order to impart fluorophilic character thereto, and represented by the nominal formula {CₙF₂ₙ₊₁—(CH₂)ₘ}; where n is an integer from 4-12, and m is an integer from 1-4. The related term “fluorous-tagged” is employed herein to refer to oligonucleotide reagents bearing one or more fluorous groups, and additionally to entire oligonucleotides synthesized with such reagents, and so bearing one or more such fluorous groups.

“Natural nucleobase” means and refers to purine and pyrimidine bases found by chemical degradation of naturally occurring nucleic acids (i.e., DNA and RNA), including adenine, guanine, hypoxanthine, xanthine, uracil, cytosine, and thymine.

“Unnatural nucleobase” means and refers to man-made analogs of natural nucleobases that may be combined with or substituted for natural nucleobases in the synthesis of modified nucleosides and oligonucleotides. Unnatural nucleobases include, by way of non-limiting example: Those wherein a H-atom has been replaced with other atoms and functional groups such as, for instance, F, Cl, Br, I, CH₃, CH₃O, NH₂, acrylic acid side chains, acrylamide side chains that contain a fluorescent tag, acrylamide side chains that contain a quencher tag, acrylamide side chains linked to biotin, etc.; aza- and deaza-versions of natural nucleobases; those wherein the point of attachment on the heterocyclic ring is a carbon atom as opposed to the nitrogen atom found in natural nucleobases. Various other synthetic modifications also yield unnatural nucleobases; the scope of heterocyclic moieties that is pertinent to the definition of an unnatural nucleobase is known to one skilled in the art of modified oligonucleotide synthesis.

“Fluorescent dye” means and refers to molecules containing two chemical functionalities: 1) that, when excited by ultraviolet light, the molecule emits light of a longer wavelength; and 2) the molecule is characterized by a reactive chemical functionality permitting attachment to other substances. Without limitation, exemplary fluorescent dyes known to those skilled in the art of oligonucleotide synthesis include: Dansyl chloride, fluorescein isothiocyanate, and tetramethylrhodamine.

“Fluorescent tag” means and refers to fluorescent dyes that when attached to a nucleoside or an oligonucleotide facilitate identification of an oligonucleotide through its fluorescent properties.

“Quencher dye” means and refers to molecules containing two chemical functionalities: 1) absorption of the light given off by nearby fluorescent materials; and 2) reactive chemical functionality permitting attachment to other substances. According to this definition, such quencher dyes may be further characterized by the transmission of light of a longer wavelength, or no light transmission, following absorption of the light given off by a nearby fluorescent material. Without limitation, exemplary quencher dyes known to those skilled in the art of oligonucleotide synthesis include tamra, dabsyl, and dabcyl

“Quencher tag” means and refers to fluorescence quenching dyes that, when attached to a nucleoside or an oligonucleotide equipped with a fluorescent tag, prohibit fluorescence if the two dyes are proximal, while permitting fluorescence if the two dyes are distant.

“Solid phase synthesis support” means and refers to an insoluble granular material upon which oligonucleotides and modified oligonucleotides are synthesized. By way of non-limiting example, solid phase synthesis supports that are well known to those skilled in the art of oligonucleotide and modified oligonucleotide synthesis include controlled pore glass (CPG), polystyrene-divinylbenzene, and polyvinylalcohol.

The following abbreviations refer to the indicated protecting groups. According to the present invention, such conventional protecting groups may be modified to the compounds and method thereof by the addition of a fluorous group, as described further herein:
“Tr” refers to the compound Ph₃C, also known as triphenylmethyl, also known as trityl.
“MMTr” refers to the compound (4-CH₃OPh)C(Ph)₂, also known as monomethoxytrityl.
“DMTr” refers to the compound (4-CH₃OPh)₂CPh, also known as dimethoxytrityl.
“TBDMS” refers to the compound t-butyldimethylsilyl.
“TES” refers to the compound triethylsilyl.
“TIPS” refers to the compound triisopropylsilyl.
“Boc” refers to the compound (CH₃)₃CO₂C, also known as t-butyloxycarbonyl.
“Cbz” refers to the compound PhCH₂O₂C, also known as benzyloxycarbonyl.
“Piv” refers to the compound (CH₃)₃CO, also known as pivaloyl.

Turning now to the following written specification and the drawings, the present invention will be understood to most generally comprise oligonucleotide reagents bearing one or more fluorous groups, incorporated either permanently or via a removable protecting group, as well as a methodology for the purification of fluorous-“tagged” oligonucleotides using separation media having greater affinity for the one or more fluorous groups of oligonucleotides synthesized from such fluorous-tagged oligonucleotide reagents than for unwanted by-products, such as, for instance, failure and deletion sequences, etc.

According to the present invention, the fluorous-tagged oligonucleotide reagents thereof may comprise protected reagents for oligonucleotide modification at the 5′-terminus, including, for example, phosphoramidites for oligonucleotide synthesis, amino-modifiers, and thiol-modifiers.

However, the incorporation of fluorous-tagged oligonucleotide reagents need not be limited to 5′ labeling of oligonucleotides, and fluorous-tagged oligonucleotide reagents consistent with the present invention may be constructed for internal labeling and 3′-labeling as well. Accordingly, it is contemplated that the fluorous-tagged oligonucleotide reagents may, in addition to comprising protected forms of conventional reagents where the protecting groups bear one or more fluorous groups, alternatively comprise reagents for permanent incorporation of the one or more fluorous groups thereof into synthetic oligonucleotides. More specific examples of such alternative reagents—that is, oligonucleotide reagents comprising at least one permanently incorporated fluorous group—are provided hereinbelow.

The fluorous-tagged oligonucleotide reagents of this invention may comprise reagents for the modification of synthetic oligonucleotides. More particularly, the reagents hereof facilitate incorporation of a fluorous-group with one or more functional groups displayed on a synthetic oligonucleotide that has been previously cleaved from the solid-phase synthesis support, in a manner not unlike that conventionally employed for the derivitization of oligonucleotides with other labels. Thus, for instance, amine- or thiol-modified oligonucleotides may be prepared using standard methods and then captured with a fluorous-acylating agent (for amine-modified oligonucleotides) or a fluorous maleimide or iodoacetamide (for thiol-modified oligonucleotides). These fluorous-tagged oligonucleotide reagents may have additional features by the incorporation of labeling moieties such as biotins, fluorophores, and/or quenchers of fluorescence, etc. Furthermore, such fluorous-tagged reagents may be selectively removable following oligonucleotide purification, or alternatively may be permanently incorporated with the oligonucleotide.

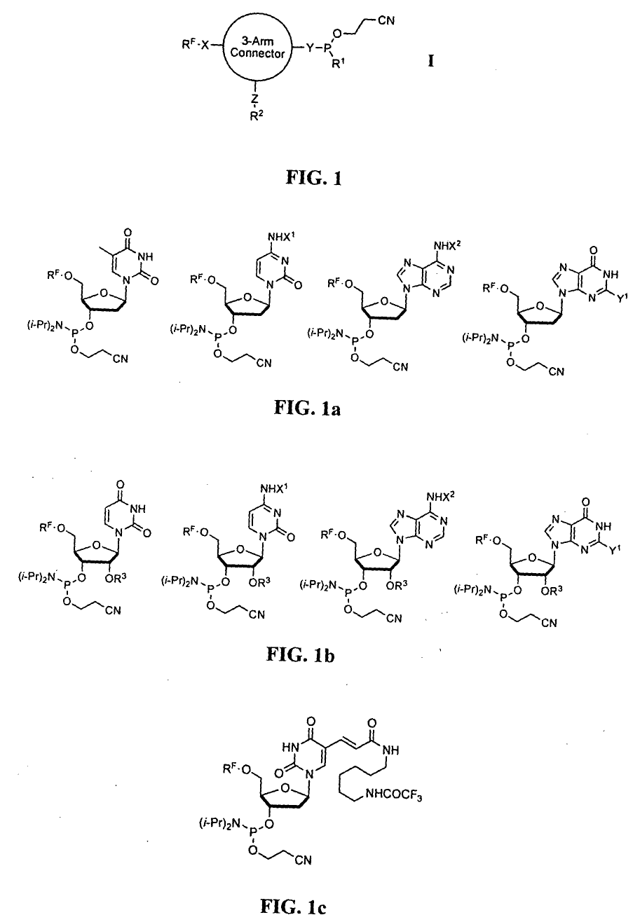

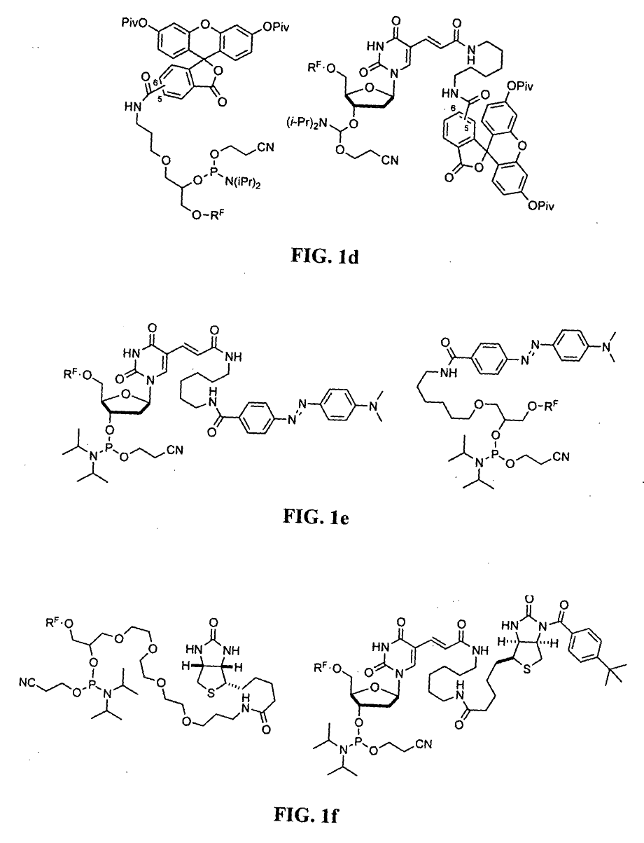

According to a first form of the present invention, the oligonucleotide reagents thereof comprise protected forms of numerous conventional reagents for oligonucleotide synthesis, including natural (i.e., DNA and RNA) phosphoramidites, unnatural phsophoramidites, fluorescent tags, quencher tags, and biotin tags. According to this first exemplary form, the inventive reagents are generically characterized by the nominal compound (I) of FIG. 1, wherein:

X is selected from the group consisting of O, N, and S;

Y is O or S;

Z is absent, or is selected from the group consisting of O, N, and S;

R¹ is selected from the group consisting of N(CH₃)₂, N(C₂H₅)₂, N(C₃H₇)₂, N(CH(CH₃)₂)₂, 1-pyrrolidinyl, 1-piperidinyl, 4-morpholinyl, and 1-imidazolyl;

R² is selected from the group consisting of a natural nucleobase, an unnatural nucleobase, a fluorescent tag, a quencher tag, biotin, and a solid phase synthesis support;

R^{F} is a fluorous protecting group selected from the group consisting of {CₙF₂ₙ₊₁—(CH₂)ₘ}DMTr, {CₙF₂ₙ₊₁—(CH₂)ₘ}MMTr, {CₙF₂ₙ₊₁—(CH₂)ₘ}Tr, {CₙF₂ₙ₊₁—(CH₂)ₘ}(Ph)₂CH, {CₙF₂ₙ₊₁—(CH₂)ₘ}PhCH₂, {CₙF₂ₙ₊₁—(CH₂)ₘ}TBDMS, {CₙF₂ₙ₊₁—(CH₂)ₘ}TES, {CₙF₂ₙ₊₁—(CH₂)ₘ}TIPS, {CₙF₂ₙ₊₁—(CH₂)ₘ}Boc, and {CₙF₂ₙ₊₁—(CH₂)ₘ}Cbz, wherein n is 4-12, m is 1-4, and R is a straight or branched alkyl of 1-4 carbon atoms; and

The three-arm connector is selected from the group consisting of: 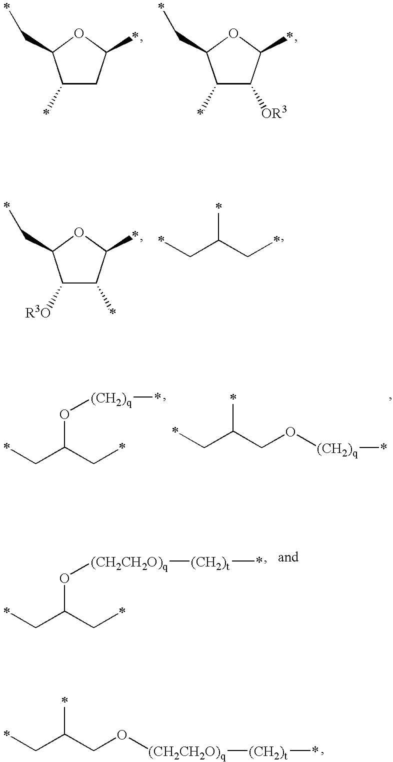
and in which group * represents attachment points for X, Y and Z, q is 2-12, t is 2-4, and R³ is selected from the group consisting of CH₃CO, (CH₃)₂CHCO, (CH₃)₂CHCH₂CO, (CH₃)₃CCO, PhCO, (CH₃)₃CSi(CH₃)₂, and (C₂H₅)₃Si.

Still more particularly, exemplary protecting groups from the foregoing category of reagents are described herein to include the following:

Protected DNA phosphoramidites according to any of the nominal compounds of FIG. 1a, wherein: X¹ is COPh or COCH₃, X² is one of the group of COPh, COi-Bu, and COCH₂OPh, Y¹ is one of the group of H, NHCOi-Bu, NHCOCH₂O(4-iPrPh), or N═CHN(CH₃)₂, and R^{F} is {CₙF₂ₙ₊₁—(CH₂)ₘ}DMTr (where n is an integer from 4-12, and m is an integer from 1-4).

Protected RNA phosphoramidites according to any of the nominal compounds of FIG. 1b, wherein: R³ is SiMe₂t-Bu or CH₂OSi(i-Pr)₃, X¹ is COPh or COCH₃, X² is one of COPh, COi-Bu, and COCH₂OPh, Y¹ is one of H, NHCOi-Bu, NHCOCH₂O(4-iPrPh), or N═CHN(CH₃)₂, R^{F} is {CₙF₂ₙ₊₁—(CH₂)ₘ}DMTr (where n is an integer from 4-12, and m is an integer from 1-4).

Protected unnatural nucleoside phosphoramidites according to the nominal compound of FIG. 1c, wherein R^{F} is {CₙF₂ₙ₊₁—(CH₂)ₘ}DMTr (where n is an integer from 4-12, and m is an integer from 1-4).

Protected fluorescent tags according to any of the nominal compounds of FIG. 1d, comprising both 5 and 6 isomers, and mixtures thereof, wherein R^{F} is {CₙF₂ₙ₊₁—(CH₂)ₘ}DMTr (where n is an integer from 4-12, and m is an integer from 1-4).

Protected quencher tags according to any of the nominal compounds of FIG. 1e, wherein R^{F} is {CₙF₂ₙ₊₁—(CH₂)ₘ}DMTr (where n is an integer from 4-12, and m is an integer from 1-4).

Protected biotin tags according to any of the nominal compounds of FIG. 1f, wherein R^{F} is {CₙF₂ₙ₊₁—(CH₂)ₘ}DMTr (where n is an integer from 4-12, and m is an integer from 1-4).

According to the foregoing exemplary compounds, and elsewhere herein where referenced, {CₙF₂ₙ₊₁—(CH₂)ₘ}DMTr more specifically comprises a conventional DMTr protecting group wherein at least one but no more than two of the hydrogen atoms have been replaced with a fluorous radical of the nominal formula {CₙF₂ₙ₊₁—(CH₂)ₘ}, where n is an integer from 4-12, and m is an integer from 1-4. Without limitation, exemplary fluorous-modified DMTr (“FDMTr”) compounds include the following: 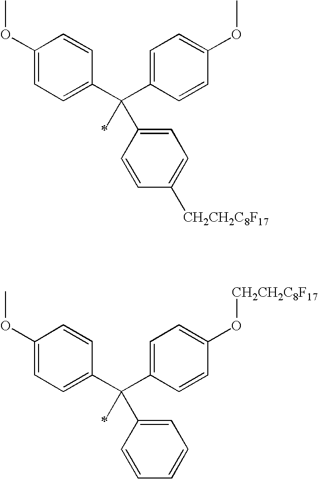
Wherein * indicates a point of attachment to O, N, or S.

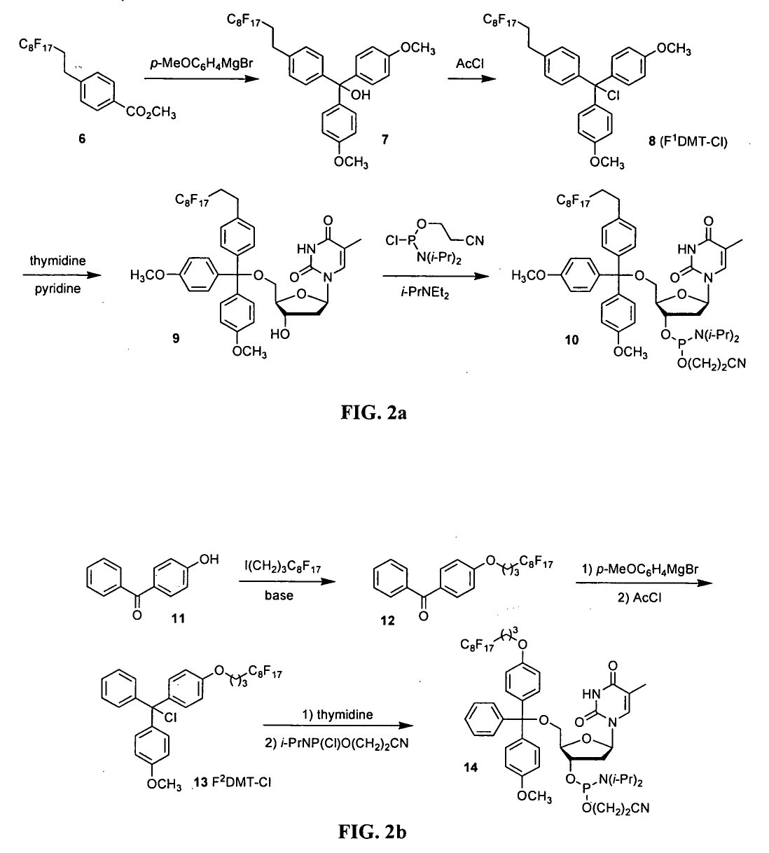

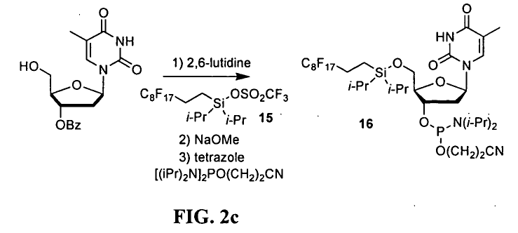

Referring now to FIGS. 2a through 2c, the following discussion details the fabrication of specific exemplary fluorous-tagged protected nucleoside phosphoramidites 10, 14, and 16 according to the nominal formula (I). More particularly, compounds 10 and 14 incorporate fluorous variations (“F¹DMTr” and “F²DMTr”, respectively) of the conventional DMTr group for 5′-protection, while compound 16 incorporates a silicon-based 5′-protecting group.

### EXAMPLE 1

### Preparation of Exemplary Fluorous-Tagged Compounds 8 and 10

In overview, the exemplary compound 10 of FIG. 2a was achieved as follows: A Grignard reaction on commercially available compound 6 (FLUOROUS TECHNOLOGIES, INC., Pittsburgh, Pa.) provided the compound F¹DMTr-OH 7, which was converted to the fluorous trityl chloride F¹DMTr-Cl 8. Fluorous tritylation of thymidine afforded compound 9, which was phosphitylated to provide cyanoethyl phosphoramidite 10. In this example, the fluorous group particularly comprises a fluorous “tail” attached via an aromatic ring carbon to the DMTr group. An ethylene spacer is used to isolate the DMTr portion of the molecule from the perfluorooctyl group in order to minimize electronic deactivation of trityl cation intermediates so that rates of tritylation/detritylation will be similar to a conventional DMTr-protecting group.

Still more particularly, a solution of methyl 4-(1H,1H,2H,2H-perfluorodecyl)benzoate 6 (4.85 g, 8.3 mmol, FLUOROUS TECHNOLOGIES, INC.) in THF (24 mL) was added over 15 min to an ice-cold solution of 4-methoxyphenylmagnesium bromide (40.6 mL of a 0.5 M solution in THF, 20.3 mmol) in dry THF (41 mL). After warming the mixture to room temperature (“rt”) for 1 h, it was poured into ice water (50 mL) and extracted with ethyl acetate (50 mL). The organic phase was dried (sodium sulfate) and concentrated in vacuo to afford 5.90 g (94%) of F¹DMTr-OH (di-(4-methoxyphenyl)-[4-( 1H,1H,2H,2H-perfluorodecyl)phenyl]methanol) 7 as a pale amber resin that crystallized upon standing. A sample was purified by chromatography on silica gel (10% ethyl acetate in hexanes), mp 103-105° C. The material was sufficiently pure to be used in the next step.

Acetyl chloride (8.25 mL, 24.6 mmol) was next added to a suspension of di-(4-methoxyphenyl)-[4-(1H,1H,2H,2H-perfluorodecyl)phenyl]methanol 7 (5.9 g, 7.7 mmol) in cyclohexane (60 mL) and the mixture heated at reflux for 1 h. After cooling to rt, the solution was concentrated to half volume in vacuo, diluted with pentane (25 mL), then cooled on an ice bath for 0.5 h. The resultant fine white crystals were collected, washed with pentane (10 mL), and dried overnight in vacuo to give 4.08 g (68%) of F¹DMTr-Cl (Di-(4-methoxyphenyl)-[4-(1H,1H,2H,2H-perfluorodecyl)phenyl]methyl chloride) 8 as a white powder, mp 136-138° C.

F¹DMTr-Cl 8 (3.18 g, 4.2 mmol) was then added over 2 h to an ice-cold solution of thymidine (605 mg, 2.5 mmol) in dry pyridine (20 mL). After warming the mixture to rt for 1 h, methanol (10 mL) was added. After stirring 0.5 h, the mixture was concentrated in vacuo and partitioned between ethyl acetate (35 mL) and water (50 mL). The organic layer was washed with brine (50 mL), dried (sodium sulfate) and concentrated to afford a golden oil, which was purified by chromatography on 130 g of silica gel (50:1 then 40:1 dichloromethane/methanol) to afford 2.06 g (84%) of 5′-O-[4,4′-Dimethoxy-4″-[4-(1H,1H,2H,2H)-perfluorodecyl]trityl]thymidine 9 as a crisp, white foam.

Next, a mixture of 5′-O-[4,4′-dimethoxy-4″-[4-(1H,1H,2H,2H)-perfluorodecyl]trityl]thymidine 9 (1.98 g, 2.03 mmol) and N,N-diisopropylethylamine (1.1 mL, 820 mg, 6.09 mmol) in anhydrous THF (70 mL) was cooled to 0° C. and treated with chloro(2-cyanoethoxy)(diisopropylamino)phosphine (680 μL, 720 mg, 3.05 mmol) in a dropwise fashion. After 5 h, the mixture was diluted with ethyl acetate (150 mL) and the resultant solution was washed with 5% aqueous NaHCO₃ (150 mL), dried over Na₂SO₄, and concentrated in vacuo at ≦30° C. Chromatography of this residue on 60 g of silica gel (previously deactivated with triethylamine, elution with 1:2 then 1:1 ethyl acetate/hexanes) gave 1.90 g (79%) of 5′-O-[4,4′-Dimethoxy-4″-[4-(1H,1H,2H,2H)-perfluorodecyl]trityl]thymidine-3′-O-(2-cyanoethyl-N,N-diisopropyl)phosphoramidite 10 as a resin whose purity was found to be >99% by HPLC.

### EXAMPLE 2

### Preparation of Exemplary Fluorous-Tagged Oligonucleotide Compound 14

The exemplary compound 14 of FIG. 2b was achieved generally as follows: A perfluoroalkyl group was attached via a propylene linker to the oxygen of a DMTr group. Alkylation of compound 11 with a fluorous iodide gave compound 12, which was subjected to a Grignard reaction and chlorination to produce alternative fluorous dimethoxytrityl chloride (“F²DMTr-Cl”) 13, which could be used to make the fluorous phosphoramidite building block 14.

Still more specifically, to a solution of 4′-hydroxybenzophenone 11 (1.57 g, 7.92 mmol) in acetonitrile (75 mL) was added 3-(perfluorooctyl)propyl iodide (5.12 g, 8.7 mmol) followed by sodium hydride (400 mg of a 60% dispersion in mineral oil, 10 mmol). After 48 h at rt, the mixture was neutralized with acetic acid and concentrated in vacuo. The residue was partitioned between ethyl acetate (200 mL) and 1% aqueous acetic acid (100 mL) and the organic phase was washed with brine (100 mL), dried over sodium sulfate, and concentrated in vacuo. The resultant residue was purified by chromatography on silica gel (10:1 hexanes/ethyl acetate) to afford 2.24 g (43%) of 4′-[(1H,1H,2H,2H,3H,3H)-Perfluoroundecyloxy]benzophenone 12.

A solution of 4-methoxyphenylmagnesium bromide (6.7 mL of a 0.5 M solution in THF, 3.34 mmol) in THF (5 mL) was then added in a dropwise fashion to an ice-cold solution of 4′-[(1H,1H,2H,2H,3H,3H)-perfluoroundecyloxy]benzophenone 12 (2.0 g, 3.03 mmol) in THF (10 mL). After 30 min, the mixture was stirred at rt overnight, then poured into 200 g of a 1:1 mixture of brine and ice. The mixture was extracted with ethyl acetate (200 mL), and the organic phase was dried over sodium sulfate and concentrated in vacuo. Chromatography of the residue (silica gel, 10:1 hexanes/ethyl acetate) gave 1.7 g (73%) of 4-Methoxy-4′-[(1H,1H,2H,2H,3H,3H)-perfluoroundecyloxy]trityl alcohol.

A solution of 4-methoxy-4′-[(1H,1H,2H,2H,3H,3H)-perfluoroundecyloxy]trityl alcohol (1.7 g, 2.22 mol), acetyl chloride (2.4 g, 30.8 mmol) and cyclohexane (20 mL) was heated at reflux for 1 h. The mixture was allowed to cool to rt and then concentrated in vacuo. The resultant residue was dissolved in toluene (30 mL), evaporated in vacuo, redissolved in toluene, then placed in a freezer overnight to afford crystals, which were collected, washed with cold toluene and pentane, then dried under high vacuum at rt to afford 0.96 g (55%) of 4-Methoxy-4′-[(1H,1H,2H,2H,3H,3H)-perfluoroundecyloxy]trityl chloride 13 as light yellow needles.

A solution of thymidine (0.24 g, 1 mmol) in dry pyridine (5 mL) was treated with 4-methoxy-4′-[(1H,1H,2H,2H,3H,3H)-perfluoroundecyloxy]trityl chloride 13 (0.96 g, 1.22 mmol). After 4 h, methanol (0.1 mL) was added, the mixture was stirred 15 min and concentrated in vacuo, and the residue was dissolved in ethyl acetate (75 mL). This solution was washed with 1:1 brine/water (40 mL), and the organic layer was dried over sodium sulfate and concentrated in vacuo. The residue was purified by chromatography on silica gel (50:1 dichloromethane/methanol containing 0.5% triethylamine) to afford 0.93 g (93%) of 5′-O-[4-Methoxy-4′-[(1H,1H,2H,2H,3H,3H)-perfluoroundecyloxy]trityl]thymidine.

A solution of 5′-O-[4-methoxy-4′-[(1H,1H,2H,2H,3H,3H)-perfluoroundecyloxy]trityl]thymidine (0.85 g, 0.86 mmol) in anhydrous dichloromethane (70 mL) was cooled to 0° C. and treated with 2-cyanoethoxy(bis-N,N-diisopropylamino)phosphine (0.35 mL, 1.67 mmol) followed by tetrazole (26 mg, 0.38 mmol). After 5 h, the mixture was diluted with dichloromethane (20 mL), and the resultant solution was washed with 5% aqueous NaHCO₃ (30 mL), dried over Na₂SO₄, and concentrated in vacuo at ≦30° C. to provide a resin that was purified by silica gel chromatography (100:1 dichloromethane/methanol containing 0.1% triethylamine) to give 0.87 g (77%) of 5′-O-[4-Methoxy-4′-[(1H,1H,2H,2H,3H,3H)-perfluoroundecyloxy]trityl]thymidine-3′-O-(2-cyanoethyl-N,N-diisopropyl)phosphoramidite 14.

### EXAMPLE 3

### Preparation of Exemplary Fluorous-Tagged Oligonucleotide Compound 16

The exemplary compound 16 of FIG. 2c was achieved by silylation of 3′-O-benzoylthymidine with fluorous silyl triflate 15, followed by debenzoylation and phosphitylation, as described more particularly hereafter:

Trifluoromethanesulfonic acid (0.26 mL, 0.44 g, 2.93 mmol) was added to ice-cold diisopropyl-(1H,1H,2H,2H-perfluorodecyl)silane (1.8 g, 3.2 mmol). After warning to rt for 16 h, the mixture was diluted with anhydrous dichloroethane (15 mL) to produce fluorous silyl triflate 15. In a separate vessel, 2,6-lutidine (0.67 mL) was added to 3′-O-benzoylthymidine (500 mg, 1.44 mmol) in anhydrous dichloroethane (15 mL) and the mixture was sonicated to uniformly suspend the undissolved solids and then added, in one portion, to the triflate 15. The reaction mixture was then warmed slightly to produce a homogenous solution. After 22 h, TLC (20:1 dichloromethane/methanol) indicated residual 3′-O-benzoylthymidine. A fresh quantity of ice-cold diisopropyl-(1H,1H,2H,2H-perfluorodecyl)silane (0.9 g) was treated with trifluoromethanesulfonic acid (0.16 mL) as outlined above, and the resultant mixture was added. After 20 h at room temperature, the reaction mixture was loaded directly onto 45 g of silica gel column packed in 50:1 dichloromethane/methanol and eluted with the same to provide (1.5 g, 91%) of 3′-O-Benzoyl-5′-O-[diisopropyl-(1H,1H,2H,2H-perfluorodecyl)silyl]thymidine.

To a stirred solution of 3′-O-benzoyl-5′-O-[diisopropyl-(1H,1H,2H,2H-perfluorodecyl)silyl]thymidine (1.4 g, 1.54 mmol) in a mixture of anhydrous methanol (20 mL) and THF (10 mL) was added 25% methanolic sodium methoxide (0.36 mL, 1.54 mmol). After 3 h at room temperature, the mixture was neutralized with Dowex 50w×8 resin (400 mesh, H⁺ form). After filtration, the filtrate was concentrated in vacuo to afford a amorphous solid that was purified by silica gel chromatography (10:1 dichloromethane/THF) to give a white solid, which was dried at 0.1 Torr at 56° C. to give 1.05 g (84%) of 5′-O-[Diisopropyl-(1H,1H,2H,2H-perfluorodecyl)silyl]thymidine.

Thereafter, a mixture of the 5′-O-[diisopropyl-(1H,1H,2H,2H-perfluorodecyl)silyl]thymidine (1.0 g,1.27 mmmol) and in anhydrous dichloromethane (20 mL) was cooled to 0° C. and treated with 2-cyanoethoxy(bis-N,N-diisopropylamino)phosphine (0.5 mL, 1.52 mmol) and tetrazole (36 mg, 0.5 mmol). After 2 h, the mixture was diluted with dichloromethane (20 mL), and the resultant solution was washed with 5% aqueous NaHCO₃ (20 mL), dried over Na₂SO₄, and concentrated in vacuo at ≦30° C. to provide a resin. This material was purified by silica gel chromatography (6:1 dichloromethane/THF containing 0.1% triethylamine) to afford 0.6 g (47%) of 5′-O-[Diisopropyl-(1H,1H,2H,2H-perfluorodecyl)silyl]thymidine-3′-O-(2-cyanoethyl-N,N-diisopropyl)phosphoramidite 16.

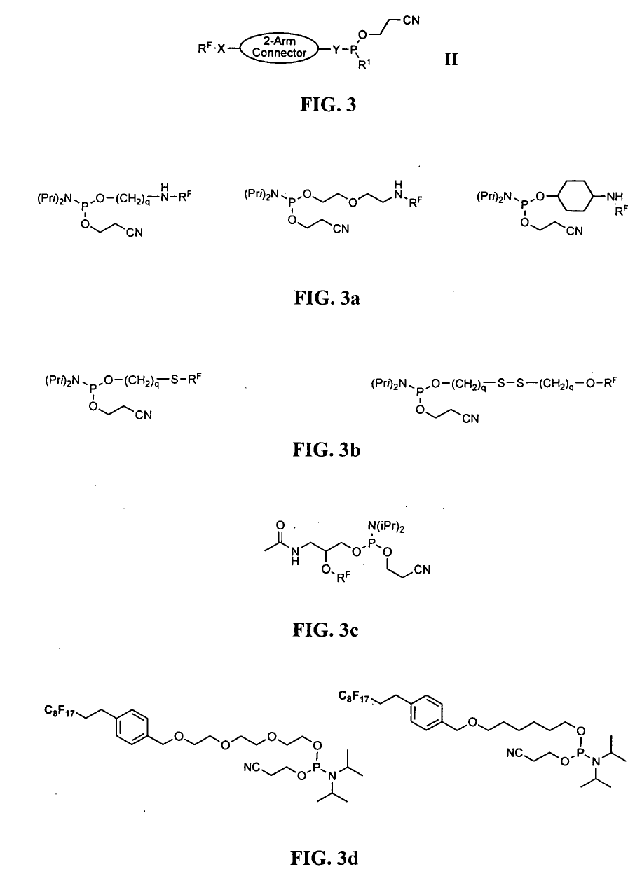

Still other conventional reagents for oligonucleotide synthesis and modification bearing fluorous tagged protecting groups according to the instant invention, including amino-modifiers, thiol-modifiers, universal fluorous phosphoramidites, and permanent fluorous tags, are, according to a second embodiment of the present invention, characterized by the nominal formula (II) of FIG. 3, in which:

X is selected from the group consisting of O, N, and S;

Y is O or S;

R¹ is selected from the group consisting of N(CH₃)₂, N(C₂H₅)₂, N(C₃H₇)₂, N(CH(CH₃)₂)₂, 1-pyrrolidinyl, 1-piperidinyl, 4-morpholinyl, and 1-imidazolyl;

R^{F} is selected from the group consisting of {CₙF₂ₙ₊₁—(CH₂)ₘ}DMTr, {CₙF₂ₙ₊₁(CH₂)ₘ}MMTr, {CₙF₂ₙ₊₁—(CH₂)ₘ}Tr, {CₙF₂ₙ₊₁—(CH₂)ₘ}(PH)₂CH, {CₙF₂ₙ₊₁—(CH₂)ₘ}PhCH₂, {CₙF₂ₙ₊₁—(CH₂)ₘ}TBDMS, {CₙF₂ₙ₊₁—(CH₂)ₘ}TES, {CₙF₂ₙ₊₁—(CH₂)ₘ}TIPS, {CₙF₂ₙ₊₁—(CH₂)ₘ}Boc, and {CₙF₂ₙ₊₁—(CH₂)ₘ}Cbz (and in which group n is 4-12, m is 1-4, and R is straight or branched alkyl of 1-4 carbon atoms); and

The two-arm connector is selected from the group consisting of *—(CH₂)_{q}—*, *—(CH₂CH₂O)_{q}—(CH₂)ₜ—*, *—(CH₂CH₂CH₂O)_{q}—(CH₂)ₜ—*, 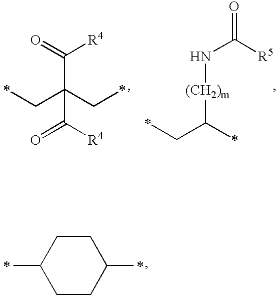
and *—(CH₂)_{q}—S—S—(CH₂)_{q}—* (and in which group * signifies attachment points for X and Y, q is 2-12, t is 2-4, m is 1-4, R⁴ is OCH₃ or NH₂, and R⁵ is selected from the group consisting of H, CF₃, CH₃, OC(CH₃)₃, and OCH₂Ph).

Exemplary compounds from the foregoing category of reagents include the following:

Amino modifiers according to any of the nominal compounds of FIG. 3a, wherein: q is an integer from 2-12; and R^{F} is selected from the group consisting of {C₈F₁₇—CH₂CH₂}DMTr, {C₈F₁₇—CH₂CH₂}MMTr, and {C₈F₁₇—CH₂CH₂}Boc.

According to the foregoing exemplary compounds, {CₙF₂ₙ₊₁—(CH₂)ₘ}Boc more specifically comprises a conventional Boc protecting group wherein at least one but no more than two of the hydrogen atoms have been replaced with a fluorous radical of the nominal formula {CₙF₂ₙ₊₁—(CH₂)ₘ}, where n is an integer from 4-12, and m is an integer from 1-4. Without limitation, exemplary fluorous-modified Boc compounds include the following:

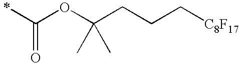

Thiol modifiers according to any of the nominal compounds of FIG. 3b, wherein: q is an integer from 2-12; and R^{F} is selected from the group consisting of {C₈F₁₇—CH₂CH₂}DMTr, {C₈F₁₇—CH₂CH₂}MMTr, and {C₈F₁₇—CH₂CH₂}Tr.

Universal fluorous phosphoramidites according to the nominal compound of FIG. 3c, wherein R^{F} is {C₈F₁₇—CH₂CH₂}DMTr.

Permanent fluorous tags according to any of the nominal compounds of FIG. 3d.

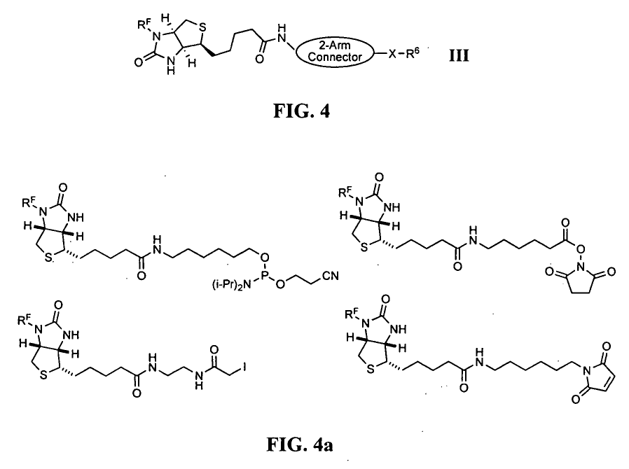

Still other oligonucleotide reagents bearing fluorous tagged protecting groups according to the instant invention, such as biotin tags for installation at the 5′-terminus, are, according to a third embodiment of the present invention, characterized by the nominal formula (III) of FIG. 4, in which:

X is selected from the group consisting of O, N and S;

R⁶ is selected from the group consisting of H, ICH₂CO—*, 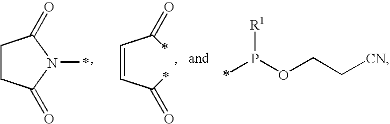
and in which group R¹ is one of N(CH₃)₂, N(C₂H₅)₂, N(C₃H₇)₂, N(CH(CH₃)₂)₂, 1-pyrrolidinyl, 1-piperidinyl, 4-morpholinyl, and 1-imidazolyl;

R^{F} is selected from the group consisting of {CₙF₂ₙ₊₁—(CH₂)ₘ}DMTr, {CₙF₂ₙ₊₁—(CH₂)ₘ}MMTr, {CₙF₂ₙ₊₁—(CH₂)ₘ}Tr, {CₙF₂ₙ₊₁—(CH₂)ₘ}(Ph)₂CH, {CₙF₂ₙ₊₁—(CH₂)ₘ}PhCH₂, {CₙF₂ₙ₊₁—(CH₂)ₘ}Boc, and {CₙF₂ₙ₊₁—(CH₂)ₘ}Cbz (and in which group n is 4-12, and m is 1-4); and

The two-arm connector is selected from the group consisting of *—(CH₂)_{q}—*, *—(CH₂)_{q}CO—* *—(CH₂CH₂O)_{q}—(CH₂)ₜ—*, *—(CH₂CH₂CH₂O)_{q}—*, 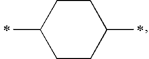
and *—(CH₂)_{q}—S—S—(CH₂)_{q}—* (and in which group * signifies attachment points for X and NH, q is 2-12, t is 2-4, R⁴ is OCH₃ or NH₂, and R⁵ is selected from the group consisting of H, CF₃, CH₃, OC(CH₃)₃, and OCH₂Ph).

As indicated, exemplary compounds from the foregoing category of reagents include biotin tags according to any of the nominal compounds of FIG. 4a, wherein R^{F} is {CₙF₂ₙ₊₁—(CH₂)ₘ}DMTr or {CₙF₂ₙ₊₁—(CH₂)ₘ}Boc (and wherein n is an integer from 4-12, and m is an integer from 1-4).

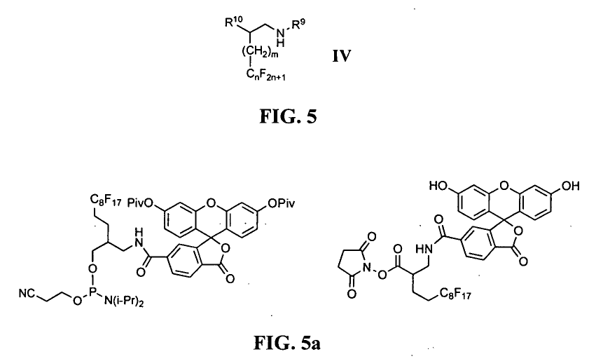

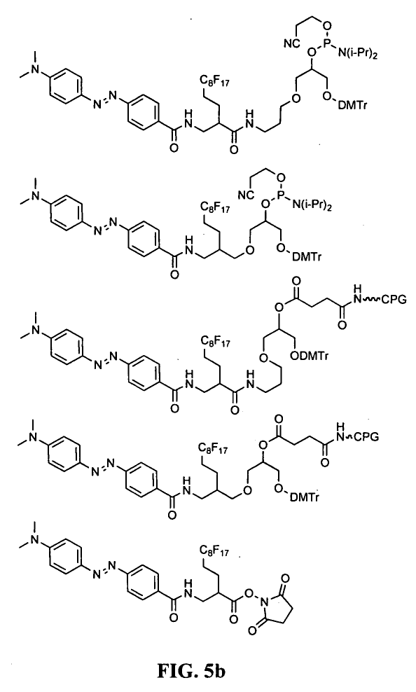

There are also provided by the present invention oligonucleotide reagents bearing permanently incorporated fluorous tags, which reagents are, in a fourth embodiment, characterized by the nominal formula (IV) of FIG. 5, in which:

n is an integer from 4-12;

m is an integer from 1-4;

R⁹ is selected from the group consisting of H, Boc, Cbz, COCH₂CH₂CO2H, a fluorescent tag, a quencher tag, biotin, and a solid phase synthesis support; and

R¹⁰ is selected from the group consisting of CO₂H, CO₂CH₃, CO₂—(N-succinimidyl), CONH(CH₂)_{q}N-maleimide, CONH(CH₂)_{q}NHCOCH₂I, CONH(CH₂)_{q}NHCOCH₂Br, CONH(CH₂)_{q}OCH₂CH(OR⁸)CH₂OR⁷, CH₂OH, CH₂OP(R¹)OCH₂CH₂CN, CH₂OCH₂CH(OR⁸)CH₂OR⁷, CH₂OCH(CH₂OR⁷) CH₂OR⁸, CH₂O(CH₂)_{q}OR⁷, CH₂O(CH₂CH₂O)_{q}R⁷, and CH₂O(CH₂)_{q}—S—S—(CH₂)_{q}OR⁷, and in which group q is an integer from 2-12, R⁷ is one of H, COCH₂CH₂CO2H, DMTr, MMTr, a solid phase synthesis support, and P(R¹)OCH₂CH₂CN, R¹ is one of N(CH₃)₂, N(C₂H₅)₂, N(C₃H₇)₂, N(CH(CH₃)₂)₂, 1-pyrrolidinyl, 1-piperidinyl, 4-morpholinyl, and 1-imidazolyl, and R⁸ is one of H, COCH₂CH₂CO2H, DMTr, MMTr, a solid phase synthesis support, and P(R¹)OCH₂CH₂CN, and when R⁷ and R⁸ are both present they are not identical.

Exemplary compounds from the foregoing category of reagents are include the following:

Fluorescent tags according to any of the nominal compounds of FIG. 5a.

Quencher tags according to any of the nominal compounds of FIG. 5b.

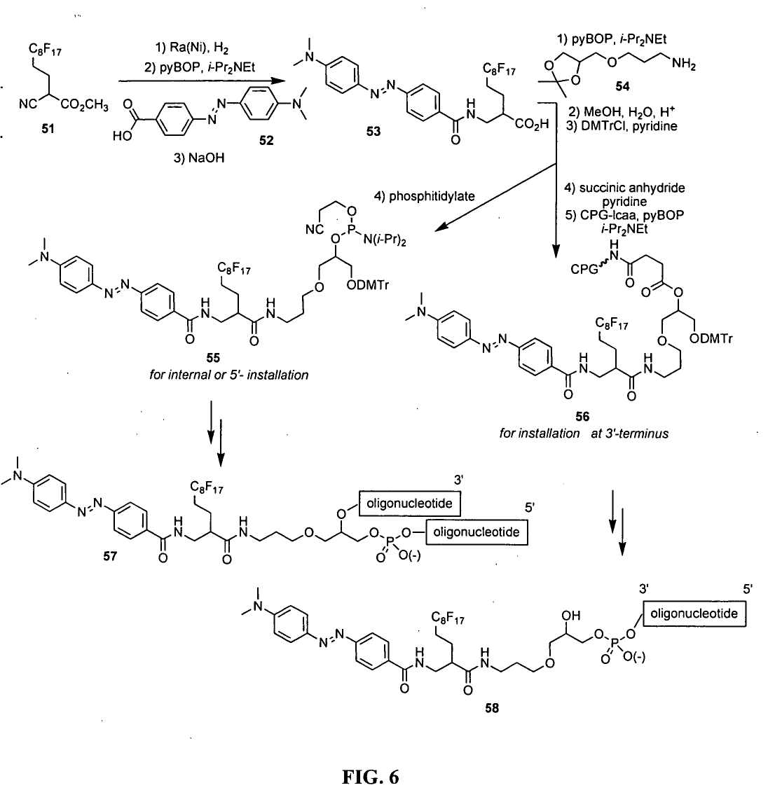

With reference now being had to FIG. 6, examples of such permanently fluorous-tagged oligonucleotide reagents that could be installed internally or at the 5′- or 3′-termini are shown to include quenchers of fluorescence such as dabcyl. The incorporation of such quenchers of fluorescence into an oligonucleotide is important in the conventional generation of fluorescent hybridization probes such as molecular beacons, and the installation of a fluorous-tagged variant of such probes would facilitate the purification thereof.

More specifically, there is illustrated in FIG. 6 the reduction of compound 51 followed by coupling with dabcyl acid 52 to yield compound 53, which was coupled with compound 54 and converted to the CPG-bound fluorous-tagged dabcyl reagent 56, which can be used to install the fluorous-tagged dabcyl group into an oligonucleotide at the 3′-position (as in compound 58) for purification purposes. Alternatively, the phosphoramidite 55 may be used to install a fluorous dabcyl group internally within an oligonucleotide or at the 5′-terminus, such as in compound 57.

The following details more particularly the fabrication of the foregoing and other exemplary compounds according to the present invention.

### EXAMPLE 4

### Preparation of Exemplary Fluorous-Tagged Oligonucleotide Compounds 55 and 56

The known compound methyl 2-cyano-5,5,6,6,7,7,8,8,9,9,10,10,11,11,12,12,12-heptadecafluorododecanoate 51 (5 g, 8.94 mmol) was dissolved in methanol (30 mL) in a Parr bottle. Raney nickel (2 g, pre-washed with methanol) and saturated methanolic ammonia (10 mL) were added and the mixture was hydrogenated at 50 psi for 24 h. Filtration of the mixture through Celite and concentration of the filtrate in vacuo gave 4.16 g (85%) of methyl 2-aminomethyl-5,5,6,6,7,7,8,8,9,9,10,10,11,11,12,12,12-heptadecafluorododecanoate as a pale yellow oil, R_{f}=0.15 (hexanes-ethyl acetate, 1:1), positive ninhydrin test.

N,N-Diisopropylethylamine (1.95 mL, 11.48 mmol) was added to a solution of methyl 2-aminomethyl-5,5,6,6,7,7,8,8,9,9,10,10,11,11,12,12,12-heptadecafluorododecanoate (3.15 g, 5.74 mmol), 4-(4-dimethylaminophenylazo)benzoic acid 52 (1.55 g, 5.74 mmol), and pyBOP (3.13 g, 6.02 mmol) in pyridine (25 mL) and dichloromethane (45 mL). After 16 h at rt, water was added and the mixture was extracted with dichloromethane. The organic phase was dried over sodium sulfate and concentrated in vacuo, then the resultant residue was purified by silica gel chromatography (4:1 hexanes/ethyl acetate) to afford 2.96 g (65%) of Methyl 2-[[4-(4-dimethylaminophenylazo)benzoylamino]methyl]-5,5,6,6,7,7,8,8,9,9,10,10,11,11,12,12,12-heptadecafluorododecanoate, R_{f} 0.75 (1:1 hexanes/ethyl acetate). UV (methanol) λₘₐₓ 430 nm; ¹H NMR (500 MHz, CDCl₃): δ 7.91 (2 H, d, J=9.0 Hz), 7.88 (2 H, d, J=8.5 Hz), 7.85 (2 H, d, J=8.5 Hz), 6.77 (2 H, d, J=9.0 Hz), 7.68 (1 H, br t, J=6.0 Hz), 3.79-3.64 (1 H, m), 3.77 (3 H, s), 3.68-3.62 (1 H, m), 3.12 (6 H, s), 2.87-2.82 (1 H, m), 2.32-2.18 (2 H, m), 2.09-2.00 (1 H, m), 1.94-1.87 (1 H, m).

Methyl 2-[[4-(4-dimethylaminophenylazo)benzoylamino]methyl]-5,5,6,6,7,7,8,8,9,9,10,10,11,11,12,12,12-heptadecafluorododecanoate (1.85 g, 2.31 mmol) was dissolved in methanol (40 mL) and 1 M aqueous sodium hydroxide (10 mL) was added. The mixture was heated at 65° C. for 18 h, cooled, diluted with water, and extracted with ethyl acetate. The organic phase was dried (Na₂SO₄) and concentrated in vacuo to give 1.77 g (97%) of 2-[[4-(4-Dimethylaminophenylazo)benzoylamino]methyl]-5,5,6,6,7,7,8,8,9,9,10,10,11,11,12,12,12-heptadecafluorododecanoic acid (53) as an orange solid, R_{f} 0.20 (ethyl acetate).

N,N-Diisopropylethylamine (755 μL, 4.44 mmol) was added to a solution of 4-(3-aminopropyloxy)methyl-2,2-dimethyl-1,3-dioxane 54 (462 mg, 2.44 mmol), 2-[[4-(4-dimethylaminophenylazo)benzoylamino]methyl]5,5,6,6,7,7,8,8,9,9,10,10,11,11,12,12,12-heptadecafluorododecanoic acid 53 (1.75 g, 2.22 mmol), and pyBOP (1.27 g, 2.44 mmol) in pyridine (40 mL). After 16 h, the mixture was diluted with ethyl acetate and the solution was washed with saturated aqueous NaHCO₃, dried (Na₂SO₄), and concentrated in vacuo. Purification of the residue by silica gel chromatography (2:1 then 1:2 hexanes/ethyl acetate) gave 2.13 g (100%) of 2-[4-(4-Dimethylaminophenylazo)benzoylamino]methyl-N-[3-(2,2-dimethyl-1,3-dioxolan-4-yl)methoxypropyl]-5,5,6,6,7,7,8,8,9,9,10,10,11,11,12,12,12-heptadecafluorododecanamide, R_{f} 0.45 (hexanes-ethyl acetate, 1:1). UV (methanol) λₘₐₓ 430 nm.

Dowex 50×8-400 (300 mg) was washed with methanol and THF and then added to a solution of 2-[4-(4-dimethylaminophenylazo)benzoylamino]methyl-N-[3-(2,2-dimethyl-1,3-dioxolan-4-yl)methoxypropyl]-5,5,6,6,7,7,8,8,9,9,10,10,11,11,12,12,12-heptadecafluorododecanamide (1.0 g, 1.04 mmol) in THF (20 mL) and 5% aqueous MeOH (20 mL). The mixture was heated at 40° C. for 18 h, then filtered, washing with methanol. The filtrate was concentrated in vacuo to give 840 mg (88%) of 2-[4-(4-Dimethylaminophenylazo)benzoylamino]methyl-N-[3-(2,3-dihydroxypropyloxy)propyl]-5,5,6,6,7,7,8,8,9,9,10,10,11,11,12,12,12-heptadecafluorododecanamide as a deep red solid, R_{f} 0.45 (ethyl acetate).

N,N-Diisopropylethylamine (164 μL, 0.94 mmol) and 4,4′-dimethoxytrityl chloride (241 mg, 0.71 mmol) were added to a solution of 2-[4-(4-dimethylaminophenylazo)benzoylamino]methyl-N-[3-(2,3-dihydroxypropyloxy)propyl]-5,5,6,6,7,7,8,8,9,9,10,10,11,11,12,12,12-heptadecafluorododecanamide (433 mg, 0.47 mmol) in acetonitrile (60 mL) at 0° C. After 15 h at rt, methanol (5 mL) was added.

After 15 min, the mixture was diluted with dichloromethane and the resultant solution was washed with saturated aqueous NaHCO₃, dried (Na₂SO₄) and concentrated in vacuo. The residue was purified by silica gel chromatography (2:1 then 1:1 hexanes/ethyl acetate) to give 239 mg (42%) of 2-[4-(4-Dimethylaminophenylazo)benzoylamino]methyl-N-[3-[2-hydroxy-3-(4,4′-dimethoxytrityloxy)propyloxy]propyl]-5,5,6,6,7,7,8,8,9,9,10,10,11,11,12,12,12-heptadecafluorododecanamide, R_{f} 0.23 (1:1 hexanes/ethyl acetate). ¹H NMR (500 MHz, CDCl₃): δ 7.90 (2 H, d, J=9.0 Hz), 7.88-7.82 (3 H, m), 7.32-6.82 (16 H, m), 3.80 (6 H, s), 3.76-3.25 (11 H, m), 3.11 (6 H, s), 2.86-2.80 (1 H, m), 2.22-1.55 (6 H, m).

A solution of 2-[4-(4-dimethylaminophenylazo)benzoylamino]methyl-N-[3-[2-hydroxy-3-(4,4′-dimethoxytrityloxy)propyloxy]propyl]5,5,6,6,7,7,8,8,9,9,10,10,11,11,12,12,12-heptadecafluorododecanamide (132 mg, 0.11 mmol), DMAP (27 mg, 0.22 mmol) and succinic anhydride (33 mg, 0.33 mmol) in pyridine (4 mL) was heated at 50° C. for 18 h. The mixture was cooled to rt, treated with ethanol (0.5 mL) for 30 min and then concentrated in vacuo. Silica gel chromatography (20:1 ethyl acetate/methanol) of the residue gave 135 mg (93%) of 2-[4-(4-Dimethylaminophenylazo)benzoylamino]methyl-N-[3-[2-succinoyloxy-3-(4,4′-dimethoxytrityloxy)propyloxy]propyl]-5,5,6,6,7,7,8,8,9,9,10,10,11,11,12,12,12-heptadecafluorododecanamide, R_{f} 0.15 (ethyl acetate-methanol, 10:1).

Pre-activated long-chain alkylamino controlled-pore glass (lcaa-CPG, 1000 angstrom, 71 μmol/g loading, 1.50 g) was gently agitated with a solution of 2-[4-(4-dimethylaminophenylazo)benzoylamino]methyl-N-[3-[2-succinoyloxy-3-(4,4′-dimethoxytrityloxy)propyloxy]propyl]-5,5,6,6,7,7,8,8,9,9,10,10,11,11,12,12,12-heptadecafluorododecanamide (130 mg, 0.10 mmol), pyBOP (52 mg, 0.10 mmol) and N,N-diisopropylethylamine (35 μL, 0.20 mmol) in pyridine (2 mL) and dichloromethane (10 mL) at rt for 3 days (“d”). The product was filtered, washing the orange solid sequentially with dichloromethane, ether, DMF and then more dichloromethane. The resultant solid was dried in vacuo to yield 1.45 g of CPG-Linked 2-[4-(4-dimethylaminophenylazo)benzoylamino]methyl-N-[3-[2-succinoyloxy-3-(4,4′-dimethoxytrityloxy)propyloxy]propyl]-5,5,6,6,7,7,8,8,9,9,10,10,11,11,12,12,12-heptadecafluorododecanamide 56. Subsequent acidic digestion and DMT analysis confirmed the substrate loading as 26 μmol/g.

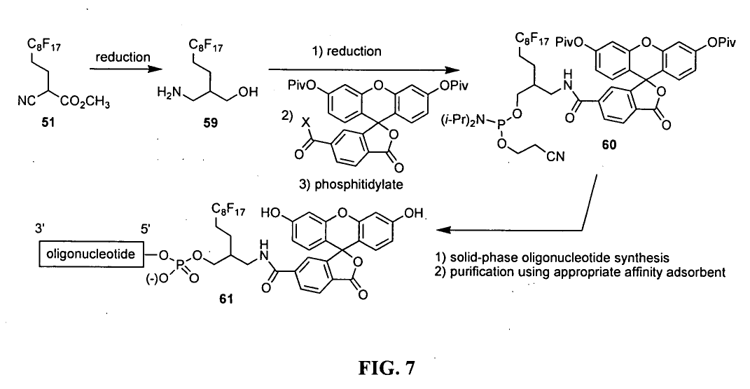

Referring next to FIG. 7, there is shown a further exemplary oligonucleotide reagent facilitating the 5′-installation of a fluorous-tagged fluorescein, according to which complete reduction of compound 51 of FIG. 6 affords compound 59, which may be coupled with (for example) 6-carboxyfluorescein and phosphitylated to give the phosphoramidite 60, a precursor of oligonucleotides 61 with a 5′-fluorous fluorophore.

Additionally, fluorous-tagged fluorophores analogous to compounds 55 and 56 (FIG. 6) would allow internal or 3′-installation (not shown).

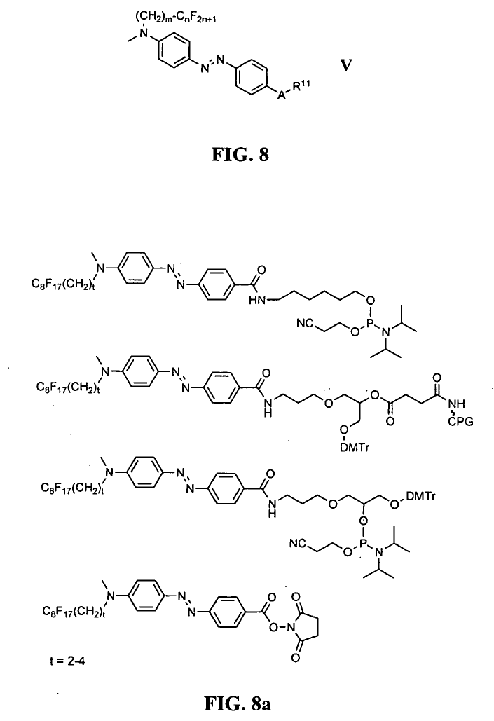

Still further oligonucleotide reagents bearing permanently incorporated fluorous tags will be seen to comprise, in a fifth embodiment of the present invention, compounds of the nominal formula (V) of FIG. 8, wherein:

m is an integer from 1-4;

n is an integer from 4-12;

A is CO or SO₂; and

R¹¹ is selected from the group consisting of Cl, OH, OCH₃, O—(N-succinimidyl), NH(CH₂)ₜOCH₂CH(OR⁸)CH₂OR⁷, NH(CH₂)_{q}OR⁷, NH(CH₂)ₜO(CH₂CH₂O)_{q}R⁷, and NH(CH₂)_{q}—S—S—(CH₂)_{q}OR⁷, and in which group R⁷ is one of H, COCH₂CH₂CO2H, DMTr, MMTr, a solid phase synthesis support, and P(R¹)OCH₂CH₂CN, R¹ is one of N(CH₃)₂, N(C₂H₅)₂, N(C₃H₇)₂, N(CH(CH₃)₂)₂, 1-pyrrolidinyl, 1-piperidinyl, 4-morpholinyl, and 1-imidazolyl, and R₈ is one of H, COCH₂CH₂CO2H, DMTr, MMTr, a solid phase synthesis support, and P(R¹)OCH₂CH₂CN, and when R⁷ and R⁸ are both present they are not identical.

Exemplary compounds from the foregoing category of reagents include quencher tags according to any of the nominal compounds of FIG. 8a, wherein t is an integer from 2-4.

It is contemplated by the inventor hereof that more than one fluorous group may be employed in any of the reagents disclosed in this specification if more demanding affinity interactions are required with the separation medium employed in subsequent purification. This can be accomplished by attachment of more than one fluorous group to one or more of the aromatic rings, or by using a fluorous group comprising one or more branched fluorous chains.

And, of course, it is envisioned that more than one of the aforedescribed fluorous-tagged oligonucleotide reagents may be incorporated into a given synthesized oligonucleotide, including for purposes of increasing affinity with the separation medium.

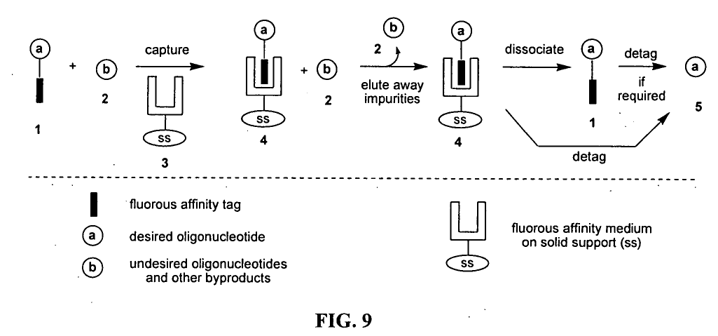

Turning now to FIG. 9, the oligonucleotide purification methodology of the instant invention is generally depicted schematically to comprise the following ordered steps:

(a) Synthesizing at least one oligonucleotide using at least one oligonucleotide reagent bearing at least one fluorous group to yield a heterogenous mixture of oligonucleotide synthesis products and reagents, said mixture including at least one target synthesized oligonucleotide bearing at least one fluorous group;

(b) passing said mixture through a separation medium having an affinity for the at least one fluorous group so that the at least one target synthesized oligonucleotide bearing at least one fluorous group is adsorbed by said separation medium;

(c) washing the separation medium with at least a first solvent to selectively dissociate therefrom substantially all synthesis products and reagents of the heterogenous mixture other than the at least one target synthesized oligonucleotide bearing at least one fluorous group; and

(d) subsequently dissociating the at least one target synthesized oligonucleotide from the separation medium, with or without the at least one fluorous group.

More particularly, and with continuing reference to FIG. 9, the heterogenous mixture of oligonucleotide synthesis products and reagents, and including the fluorous-tagged oligonucleotide 1, is passed through a cartridge or column containing an adsorbent that bears fluorous affinity groups on a solid support 3, leading to the capture of the fluorous-tagged oligonucleotide to yield the complex 4. The undesired materials 2 lacking fluorous-tagged oligonucleotides interact with the adsorbent minimally, so that washing the adsorbent with at least a first suitable solvent will remove them, leaving only the complex 4. Dissociation of the desired fluorous-tagged oligonucleotide 1 from the adsorbent may then be accomplished by washing with a second, more fluorophilic solvent.

In cases where the fluorous-group is to be retained with the synthesized oligonucleotide, the fluorous-tagged oligonucleotide 1 is the final purified target compound.

Alternatively, the fluorous-group can be removed from the target oligonucleotide 1, such as, in the case of an oligonucleotide synthesized from an oligonucleotide reagent comprising a protecting group bearing the at least one fluorous group (e.g., yielding a fluorous-tagged nucleoside positioned at the 5′ terminus), by reaction with a suitable cleaving agent to provide a purified oligonucleotide 5. This may be accomplished either after elution of the fluorous-tagged oligonucleotide 1, or while the fluorous-tagged oligonucleotide is retained on the separation medium.

As indicated, the separation medium comprises fluorous affinity groups, which may include any groups demonstrating a stronger interaction with the fluorous-group of the oligonucleotide reagents of the present invention. Thus, it is contemplated that the separation medium may take the form of conventional lipophilic reverse-phase adsorbents based on a matrix of silica, poly(divinylbenzene) or polystyrene cross-linked with divinylbenzene.

However, it is even more preferred that the separation medium comprise a reverse-phase adsorbent bearing fluorinated groups, including, for example, a polymeric (such as, for instance, poly(divinylbenzene) or polystyrene cross-linked with divinylbenzene) or silica matrix bearing fluorinated organic groups.

The following experimental examples further demonstrate the foregoing methodology using fluorous-tagged oligonucleotide reagents as described elsewhere herein.

### EXPERIMENTAL EXAMPLE 5

### Synthesis and Purification of Fluorous-Tagged Oligonucleotides

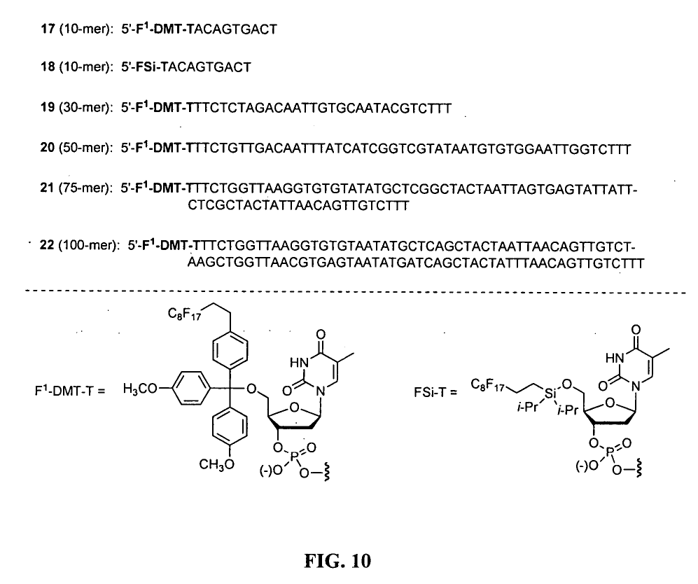

Oligonucleotides were prepared on an EXPEDITE 8909 synthesizer using standard 2-cyanoethyl N,N-diisopropylphosphoramidite chemistry. The syntheses were carried out on either 0.2 μmol or 1 μmol scale using 1000 angstrom CPG solid supports bearing a 3′-linked 5′-O-DMTr-thymidine, with the exception of 100-mer synthesis, which was carried out on 2000 angstrom support. In addition to the fluorous-tagged nucleoside phosphoramidites 10, 14, and 16 (FIGS. 2a-2c), 5′-O-DMTr-dA^{Bz}, -dC^{Ac}, and -dG^{iBu} nucleoside phosphoramidites were used. Reagents recommended by the manufacturer were employed, with the exception of the substitution of THF/pyridine/Ac₂O for Cap A and 16% N-methylimidazole/THF for Cap B. The manufacturer's protocols and coupling times were used (fluorous phosphoramidites were coupled for 15 min using standard “X” protocol), except that capping was performed for 75 s. Syntheses were performed in the trityl-on mode, and the oligonucleotides were cleaved from the CPG support with 3 mL of concentrated ammonium hydroxide at rt for 1 h. Nucleobase deprotection was accomplished by heating the resultant ammonium hydroxide solution at 55° C. for 16-24 h. In addition to various control oligonucleotides bearing a 5′-DMTr group or a free 5′-hydroxyl group, a variety of fluorous-tagged oligonucleotides were prepared using the nucleodies phosphoramidites 10, 14, and 16 (FIGS. 2a-2c). Among them were the sequences of FIG. 10, wherein F¹DMTr and FSi-T equate to the following compounds:

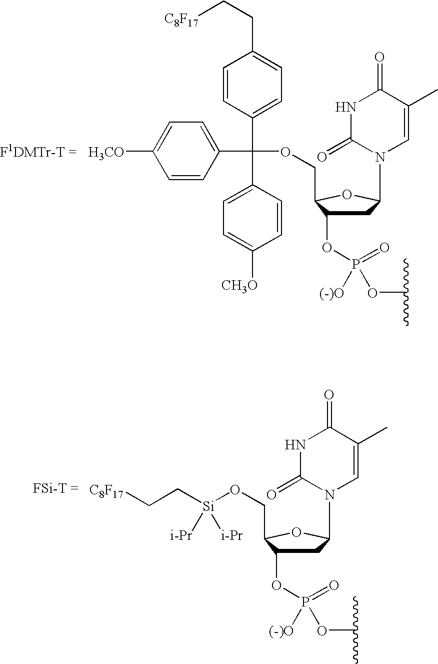

To illustrate the use of fluorous “tagging” to purify oligonucleotides, nucleoside phosphoramidites 10 and 16 (FIGS. 2a and 2c) were used to install a fluorous-bearing thymidine monomer at the 5′-terminus of several oligodeoxyribonucleotides 17-22 ranging in length from 10-100 nucleotides (FIG. 10). Standard solid-phase synthesis chemistry was used to prepare these materials except that the final acid deblocking step was not carried out. Cleavage of the oligonucleotides from the solid support and nucleobase deblocking with ammonium hydroxide was carried out as usual, affording the desired oligonucleotides as crude mixtures with the commonly observed by-products (failure sequences, benzamide, etc.).

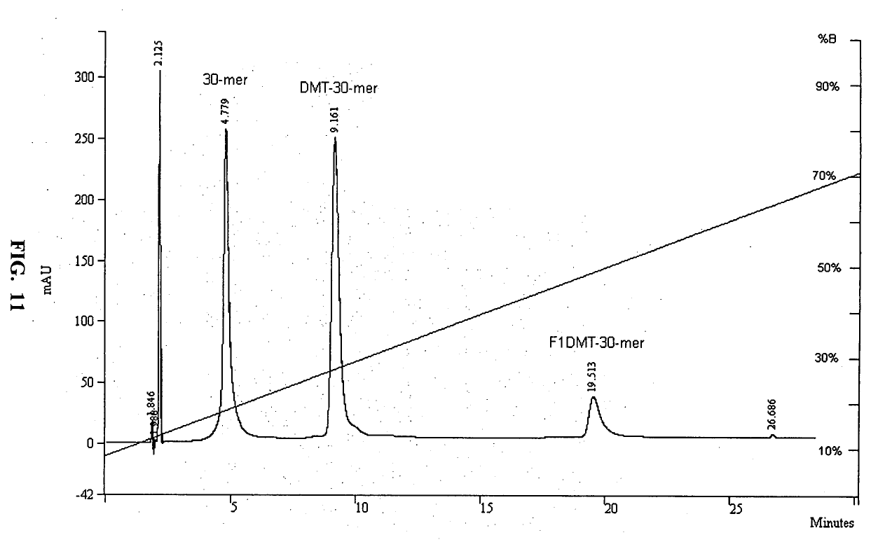

HPLC analysis of these crude oligonucleotide mixtures showed that the fluorous-tagged full-length oligonucleotides are highly retained on a fluorous HPLC adsorbent. To illustrate the magnitude of retention, the fluorous 30-mer oligonucleotide 19 was mixed with samples of the corresponding non-fluorous 5′-DMTr-on and DMTr-off oligonucleotides (prepared separately) and injected onto a fluorous silica HPLC column (FLUOROFLASH 4.6×150 mm, mobile phase A=0.1 M triethylammonium acetate, mobile phase B=acetonitrile). FIG. 11 shows that the fluorous-tagged material 19 is strongly retained over the corresponding DMTr-on and DMTr-off 30-mers, eluting when the acetonitrile percentage neared. 50% in the gradient profile. Note that an isocratic elution gave even larger differences in retention times. A key observation from this experiment is that, in relation to a DMTr-off oligonucleotide, the F¹-DMTr on oligonucleotide is retained much more strongly than a DMTr-on oligonucleotide.

The utility of the fluorous method for oligonucleotide purification was further illustrated using solid-phase extraction (“SPE” or “cartridge purification”).

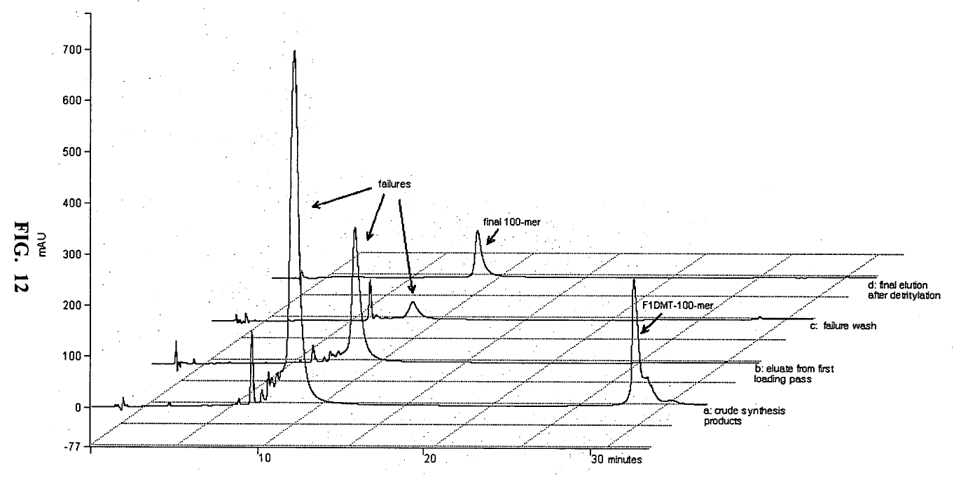

In a first example, the 100-mer oligonucleotide 22 was prepared on a 0.2 micromole scale using standard solid-phase synthesis techniques on 2000 angstrom CPG support using the protected nucleoside phosphoramidite 10 (FIG. 2a) to install a fluorous DMTr thymidine (“F¹DMTr-T”) at the 5′ terminus. Cleavage from the support with ammonium hydroxide at room temperature followed by deblocking the nucleobases with ammonium hydroxide at 55° C. gave a solution of the crude products in ammonium hydroxide solution. HPLC analysis of this mixture showed the F¹DMTr-100-mer well-separated from failure sequences and other synthesis by-products (trace (a) in FIG. 12), while UV measurement at 260 nm indicated that about 6 optical density units (ODU) of the total mixture could be assigned to the fluorous-tagged 100-mer 22.

Without removing the ammonia used in the deblocking step, the crude deprotected oligonucleotide 22 (0.2 μmol scale) was diluted with an equal volume of loading buffer, following which the resultant solution was passed through a pre-conditioned FLUORO-PAK (BERRY & ASSOCIATES, Dexter, Mich.) column containing 100 mg of a pH-stable, fluorinated polymeric adsorbent at a flow rate of 5 drops/s with pressure from a disposable PE/PP syringe or a compressed gas line (air or inert gas), or using vacuum via a commercial vacuum box. To pre-condition the column, the following were passed therethrough to waste: 2 mL of acetonitrile, 2 mL of 0.1 M TEAA (triethylammonium acetate), and 2 mL of loading buffer (100 mg/mL NaCl in water containing 5% N,N-dimethylformamide).

The eluate was collected and analyzed by HPLC (trace (b) of FIG. 12), showing that the fluorous oligonucleotide had been fully retained by the adsorbent, whereas a substantial amount of the non-fluorous-tagged materials had eluted. Other experiments showed that a loading rate of 2 drops/s could be employed, which loaded most of the fluorous oligonucleotide. In those cases, passing the eluate through the tube a second time assured complete binding.

2 mL of 10% acetonitrile in 0.1 M TEAA followed by 2 mL of water was then passed through the column. The combined eluates were collected and analyzed by HPLC (trace (c) of FIG. 12), showing that failure sequences were eluted without stripping the fluorous-tagged oligonucleotide from the column. In a separate experiment, two 2 mL elutions with 10% acetonitrile in 0.1 M TEAA were carried out before the water wash to verify that the failure sequences were entirely removed in the first 2 mL elution (trace not shown in FIG. 12).

3 mL of 3% aqueous TFA (trifluoroacetic acid) was passed through the tube to waste, followed by 1 mL of 0.1 M TEAA and 1 mL of water. Introduction of the aqueous trifluoroacetic acid to the column caused detritylation of the bound fluorous 100-mer.

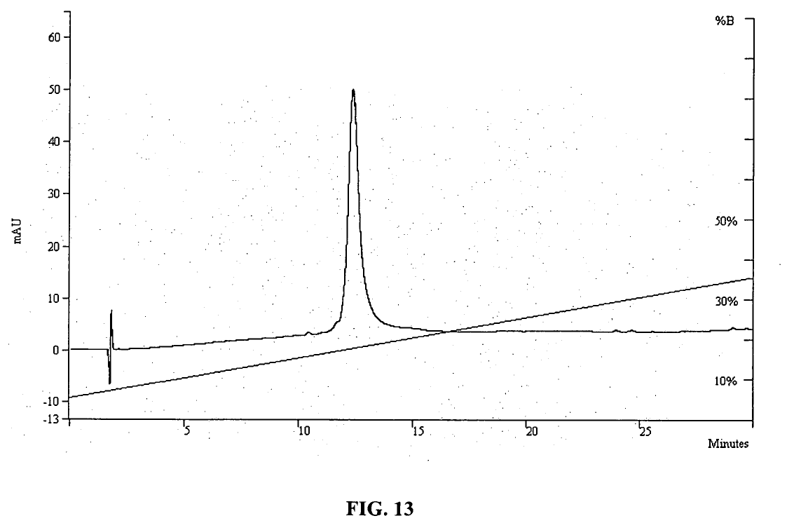

Final elution of the trityl-off, fully deprotected 100-mer was accomplished with 1 mL of 10% aqueous acetbnitrile in water passed through the column, collecting the eluate in an Eppendorf tube. Other experiments showed that a smaller volume (about 600 μL) could be used if 20-30% aqueous acetonitrile was used instead. HPLC analysis showed largely a single peak for the resultant 100-mer (trace (d) in FIGS. 12 and 13). UV measurement at 260 nm showed that 6 ODU of purified oligonucleotide was obtained, representing an approximately quantitative recovery of the detritylated 100-mer.

Other runs with several different 0.2 μmol synthesis batches of fluorous-tagged 100-mer 22 gave 2.7-6 ODU of purified detritylated 100-mer, representing approximately 70-100% recovery based on estimation of the maximum amount of fluorous-tagged 100-mer 22 estimated in the crude mixtures, an unexpectedly high recovery.

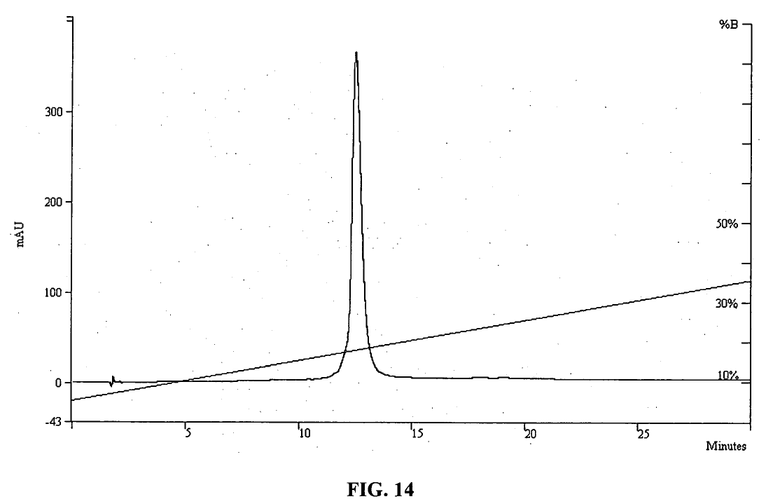

Similarly conducted fluorous purification and on-column detritylation of multiple synthesis batches of the 30-, 50-, and 75-mers 19-21 led to 60-100% recovery of maximum estimated amount of oligonucleotides based on HPLC analysis of the amount of 19-21 present in the crude synthesis products. As another example, the HPLC trace of the fluorous-purified 75-mer oligonucleotide derived from on-column detritylation of 21 is shown in FIG. 14.

In each case, the amount of product recovered was found to be a substantial fraction (typically 60-90+%) of the amount theoretically available as estimated by the area percentage of the F¹DMTr-oligonucleotide peak in the HPLC of the crude synthesis product. This is in contrast to typical DMTr-on cartridge purifications, which suffer from low yield and purity with oligonucleotides longer than 30-40 nucleotides.

While the fluorous purification technique of the present invention is surprisingly effective for isolating full-length material without contamination by failure sequences, it is recognized that the fluorous-purified material is still a distribution of the full-length product plus the expected deletion oligonucleotides (i.e., n-1, n-2, etc.), since the final phosphoramidite coupling attaches a fluorous-tagged nucleotide to a preexisting distribution of the desired chain plus deletion materials. These deletions are not resolved by HPLC, but can be detected by capillary electrophoresis analysis.

In other examples, alternate adsorbents were found to allow the purification of fluorous-tagged oligonucleotides, although yields and purities were not as desirable. Nonetheless, the RP adsorbent should find use in the analysis and purification of fluorous tagged oligonucleotides in some cases. Exemplary alternate adsorbents include FLUOROFLASH (Fluorous Technologies, Inc.), a silica-based material bearing fluorinated groups, could be used provided that the ammonia from the deprotection solution was removed in order to avoid degradation of the silica matrix. Other examples include POLY-PAK (Glen Research Corporation) and OPC (Applied Biosystems, Inc.) cartridges, which use polymeric reverse-phase adsorbents, allowing direct loading of the ammonia solutions.

Of course, the foregoing is merely illustrative of the present invention, and those of ordinary skill in the art will appreciate that many additions and modifications to the present invention, as set out in this disclosure, are possible without departing from the spirit and broader aspects of this invention as defined in the appended claims.

The invention in which an exclusive property or privilege is claimed is defined as follows:

1. A method for the purification of oligonucleotides, comprising the following ordered steps:
(a) Synthesizing at least one oligonucleotide using at least one oligonucleotide reagent bearing at least one fluorous group to yield a heterogenous mixture of oligonucleotide synthesis products and reagents, said mixture including at least one target synthesized oligonucleotide bearing at least one fluorous group;
(b) passing said mixture through a separation medium having an affinity for the at least one fluorous group so that the at least one target synthesized oligonucleotide bearing at least one fluorous group is adsorbed by said separation medium;
(c) washing the separation medium with at least a first solvent to selectively dissociate therefrom substantially all synthesis products and reagents of the heterogenous mixture other than the at least one target synthesized oligonucleotide bearing at least one fluorous group; and
(d) subsequently dissociating the at least one target synthesized oligonucleotide from the separation medium, with or without the at least one fluorous group.

2. The method of claim 1, wherein the at least one oligonucleotide reagent comprises a protected nucleoside the protecting group of which bears the at least one fluorous group, the at least one target synthesized oligonucleotide comprises the protected nucleoside, and wherein further the step (d) comprises removing from the at least one target synthesized oligonucleotide the protecting group bearing the at least one fluorous group, and thereafter eluting said at least one target synthesized oligonucleotide from the separation medium without the protecting group bearing the at least one fluorous group.

3. The method of claim 2, wherein the at least one target synthesized oligonucleotide bearing at least one fluorous group comprises, at the 5′ terminus thereof, a single protected nucleoside the protecting group of which bears at least one fluorous group.

4. The method of claim 1, wherein the step (d) comprises washing the separation medium with at least a second solvent more fluorophilic than said at least first solvent to dissociate from said separation medium the at least one target synthesized oligonucleotide bearing at least one fluorous group.

5. The method of claim 4, wherein the at least one oligonucleotide reagent comprises a protected nucleoside the protecting group of which bears the at least one fluorous group, the at least one target synthesized oligonucleotide comprises the protected nucleoside, and wherein the method comprises the further ordered step (e) of removing from the at least one target synthesized oligonucleotide the protecting group bearing the at least one fluorous group.

6. The method of claim 5, wherein the at least one target synthesized oligonucleotide bearing at least one fluorous group comprises, at the 5′ terminus thereof, a single protected nucleoside the protecting group of which bears at least one fluorous group.

7. The method of claim 1, wherein said separation medium comprises fluorous affinity groups.

8. The method of claim 7, wherein said separation medium comprises a reverse-phase adsorbent bearing fluorinated groups.

9. The method of claim 7, wherein said separation medium comprises a polymeric matrix bearing fluorinated oligonucleotide groups.

10. The method of claim 9, wherein said polymeric matrix is chosen from poly(divinylbenzene) or polystyrene cross-linked with divinylbenzene.

11. The method of claim 7, wherein said separation medium comprises a silica matrix bearing fluorinated groups.

12. The method of claim 7, wherein said separation medium is a lipophilic reverse-phase adsorbent based on a matrix of silica, poly(divinylbenzene) or polystyrene cross-linked with divinylbenzene.

13. An oligonucleotide reagent bearing at least one fluorous group.

14. The oligonucleotide reagent of claim 12, characterized by the following nominal formula (I): 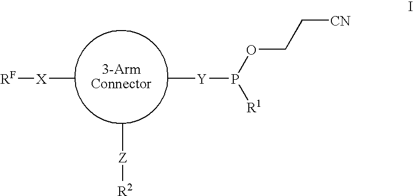
Wherein,
X is selected from the group consisting of O, N, and S;
Y is O or S;
Z is absent, or is selected from the group consisting of O, N, and S;
R¹ is selected from the group consisting of N(CH₃)₂, N(C₂H₅)₂, N(C₃H₇)₂, N(CH(CH₃)₂)₂, 1-pyrrolidinyl, 1-piperidinyl, 4-morpholinyl, and 1-imidazolyl;
R² is selected from the group consisting of a natural nucleobase, an unnatural nucleobase, a fluorescent tag, a quencher tag, biotin, and a solid phase synthesis support;
R^{F} is a fluorous protecting group selected from the group consisting of {CₙF₂ₙ₊₁—(CH₂)ₘ}DMTr, {CₙF₂ₙ₊₁—(CH₂)ₘ}MMTr, {CₙF₂ₙ₊₁—(CH₂)ₘ}Tr, {CₙF₂ₙ₊₁—(CH₂)ₘ}(Ph)₂CH, {CₙF₂ₙ₊₁—(CH₂)ₘ}PhCH₂, {CₙF₂ₙ₊₁—(CH₂)ₘ}TBDMS, {CₙF₂ₙ₊₁—(CH₂)ₘ}TES, {CₙF₂ₙ₊₁—(CH₂)ₘ}TIPS, {CₙF₂ₙ₊₁—(CH₂)ₘ}Boc, and {CₙF₂ₙ₊₁—(CH₂)ₘ}Cbz, wherein n is 4-12, m is 1-4, and R is a straight or branched alkyl of 1-4 carbon atoms; and 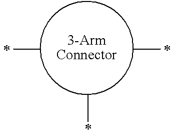
is selected from the group consisting of 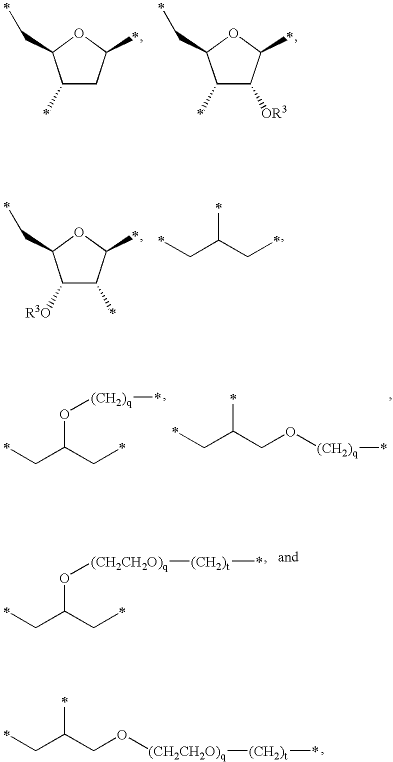
wherein * represents attachment points for X, Y and Z, q is 2-12, t is 2-4, and R³ is selected from the group consisting of CH₃CO, (CH₃)₂CHCO, (CH₃)₂CHCH₂CO, (CH₃)₃CCO, PhCO, (CH₃)₃CSi(CH₃)₂, and (C₂H₅)₃Si.

15. The oligonucleotide reagent of claim 14, wherein said reagent comprises a DNA phosphoramidite according to any of the following nominal compounds: 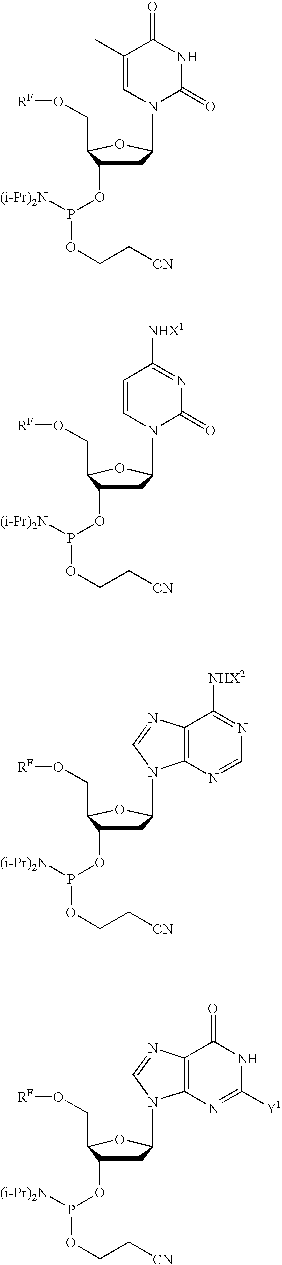
Wherein,
X¹ is COPh or COCH₃
X² is selected from the group consisting of COPh, COi-Bu, and COCH₂OPh
Y¹ is selected from the group consisting of H, NHCOi-Bu, NHCOCH₂O(4-iPrPh), or N═CHN(CH₃)₂; and
R^{F} is {CₙF₂ₙ₊₁—(CH₂)ₘ}DMTr.

16. The oligonucleotide reagent of claim 14, wherein said reagent comprises a RNA phosphoramidite according to any of the following nominal compounds: 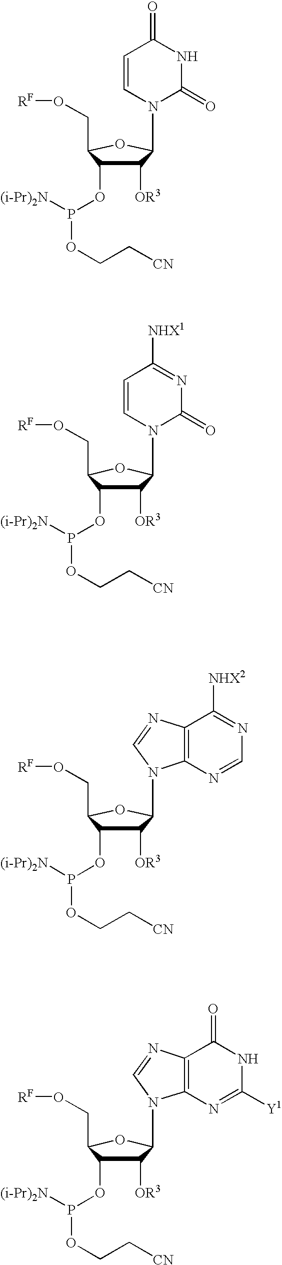
Wherein,
R³ is SiMe₂t-Bu or CH₂OSi(i-Pr)₃
X¹ is COPh or COCH₃
X² is selected from the group consisting of COPh, COi-Bu, and COCH₂OPh
Y¹ is selected from the group consisting of H, NHCOi-Bu, NHCOCH₂O(4-iPrPh), or N═CHN(CH₃)₂; and
R^{F} is {CₙF₂ₙ₊₁—(CH₂)ₘ}DMTr.

17. The oligonucleotide reagent of claim 14, wherein said reagent comprises an unnatural nucleoside phosphoramidite according to the following nominal compound: 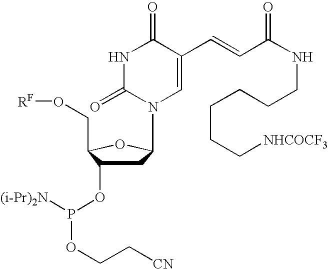
Wherein,
R^{F} is {CₙF₂ₙ₊₁—(CH₂)ₘ}DMTr.

18. The oligonucleotide reagent of claim 14, wherein said reagent comprises a fluorescent tag according to any of the following nominal compounds comprising both 5 and 6 isomers, and mixtures thereof. 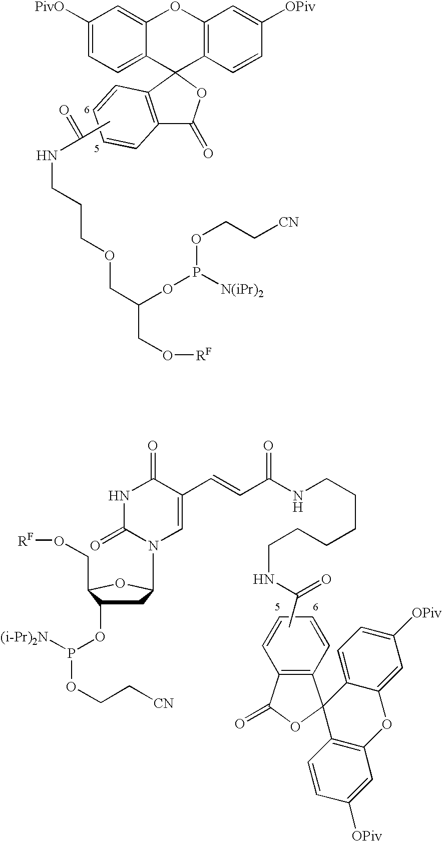
Wherein,
R^{F} is {CₙF₂ₙ₊₁—(CH₂)ₘ}DMTr.

19. The oligonucleotide reagent of claim 14, wherein said reagent comprises a quencher tag according to any of the following nominal compounds: 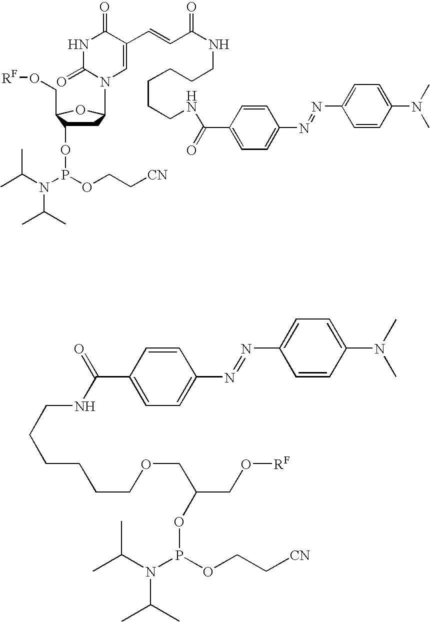
Wherein,
R^{F} is {CₙF₂ₙ₊₁—(CH₂)ₘ}DMTr.

20. The oligonucleotide reagent of claim 14, wherein said reagent comprises a biotin tag according to any of the following nominal compounds: 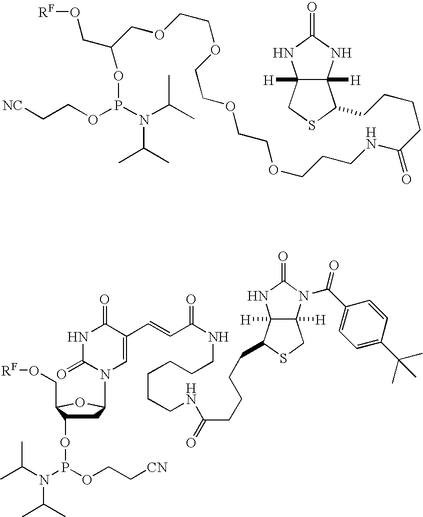
Wherein,
R^{F} is {CₙF₂ₙ₊₁—(CH₂)ₘ}DMTr.

21. The oligonucleotide reagent of claim 13, characterized by the following nominal formula (II): 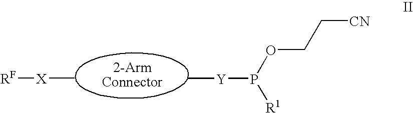
Wherein,
X is selected from the group consisting of O, N, and S;
Y is O or S;
R¹ is selected from the group consisting of N(CH₃)₂, N(C₂H₅)₂, N(C₃H₇)₂, N(CH(CH₃)₂)₂, 1-pyrrolidinyl, 1-piperidinyl, 4-morpholinyl, and 1-imidazolyl;
R^{F} is selected from the group consisting of {CₙF₂ₙ₊₁—(CH₂)ₘ}DMTr, {CₙF₂ₙ₊₁—(CH₂)ₘ}MMTr, {CₙF₂ₙ₊₁—(CH₂)ₘ}Tr, {CₙF₂ₙ₊₁—(CH₂)m}(Ph)₂CH, {CₙF₂ₙ₊₁—(CH₂)ₘ}PhCH₂, {CₙF₂ₙ₊₁—(CH₂)ₘ}TBDMS, {CₙF₂ₙ₊₁—(CH₂)ₘ}TES, {CₙF₂ₙ₊₁—(CH₂)ₘ}TIPS, {CₙF₂ₙ₊₁—(CH₂)ₘ}Boc, and {CₙF₂ₙ₊₁—(CH₂)ₘ}Cbz, wherein n is 4-12, m is 1-4, and R is straight or branched alkyl of 1-4 carbon atoms; and 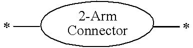
is selected from the group consisting of *—(CH₂)_{q}—*, *—(CH₂CH₂O)_{q}—(CH₂)ₜ—*, *—(CH₂CH₂CH₂O)_{q}—(CH₂)ₜ—*, 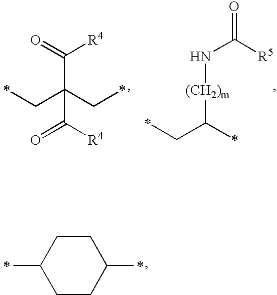
and *—(CH₂)_{q}—S—S—(CH₂)_{q}—*, wherein * signifies attachment points for X and Y, q is 2-12, t is 2-4, m is 1-4, R⁴ is OCH₃ or NH₂, and R⁵ is selected from the group consisting of H, CF₃, CH₃, OC(CH₃)₃, and OCH₂Ph.

22. The oligonucleotide reagent of claim 21, wherein said reagent comprises an amino modifier according to any of the following nominal compounds: 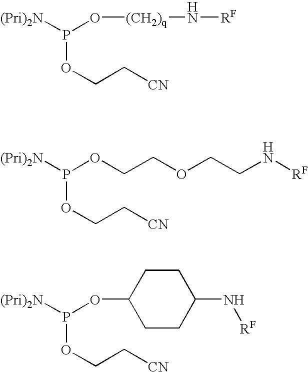
Wherein,
q is an integer from 2-12
R^{F} is selected from the group consisting of {C₈F₁₇—CH₂CH₂}DMTr, {C₈F₁₇—CH₂CH₂}MMTr, and {C₈F₁₇—CH₂CH₂}Boc

23. The oligonucleotide reagent of claim 21, wherein said reagent comprises a thiol modifier according to any of the following nominal compounds: 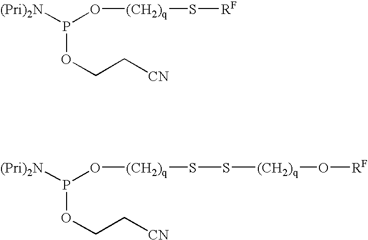
Wherein,
q is an integer from 2-12; and
R^{F} is selected from the group consisting of {C₈F₁₇—CH₂CH₂}DMTr, {C₈F₁₇—CH₂CH₂}MMTr, and {C₈F₁₇—CH₂CH₂}Tr.

24. The oligonucleotide reagent of claim 21, wherein said reagent comprises a universal fluorous phosphoramidite according to the following nominal compound: 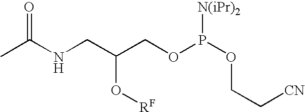
Wherein,
R^{F} is {C₈F₁₇—CH₂CH₂}DMTr.

25. The oligonucleotide reagent of claim 21, wherein said reagent comprises a permanent fluorous tag according to any of the following nominal compounds:

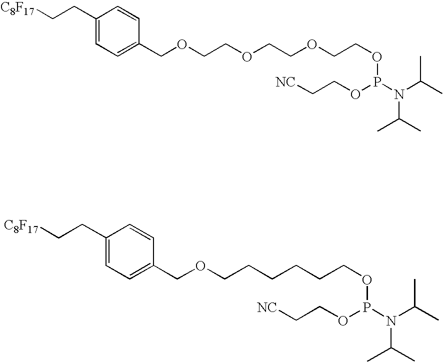

26. The oligonucleotide reagent of claim 13, characterized by the following nominal formula (III): 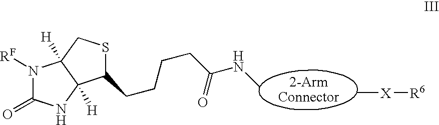
Wherein,
X is selected from the group consisting of O, N and S,
R⁶ is selected from the group consisting of H, ICH₂CO—*, 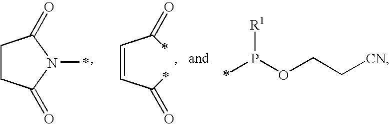
wherein R¹ is N(CH₃)₂, N(C₂H₅)₂, N(C₃H₇)₂, N(CH(CH₃)₂)₂, 1-pyrrolidinyl, 1-piperidinyl, 4-morpholinyl, and 1-imidazolyl;
R^{F} is selected from the group consisting of {CₙF₂ₙ₊₁—(CH₂)ₘ}DMTr, {CₙF₂ₙ₊₁—(CH₂)ₘ}MMTr, {CₙF₂ₙ₊₁—(CH₂)ₘ56 Tr, {CₙF₂ₙ₊₁—(CH₂)ₘ}(Ph)₂CH, {CₙF₂ₙ₊₁—(CH₂)ₘ}PhCH₂, {CₙF₂ₙ₊₁—(CH₂)ₘ}Boc, and {CₙF₂ₙ₊₁—(CH₂)ₘ}Cbz, wherein n is 4-12, and m is 1-4; and 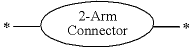
is selected from the group consisting of *—(CH₂)_{q}—, *—(CH₂)_{q}CO—* *—(CH₂CH₂O)_{q}—(CH₂)ₜ—*, *—(CH₂CH₂CH₂O)_{q}—*, 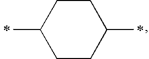
and *—(CH₂)_{q}—S—S—(CH₂)_{q}—*, wherein * signifies attachment points for X and NH, q is 2-12, t is 2-4, R⁴ is OCH₃ or NH₂, and R⁵ is selected from the group consisting of H, CF₃, CH₃, OC(CH₃)₃, and OCH₂Ph.

27. The oligonucleotide reagent of claim 26, wherein said reagent comprises a biotin tag according to any of the following nominal compounds: 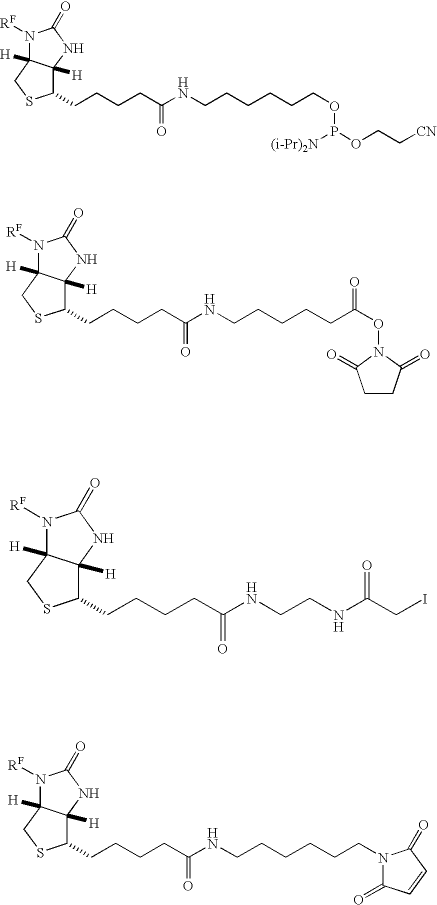
Wherein,
R^{F} is {CₙF₂ₙ₊₁—(CH₂)ₘ}DMTr or {CₙF₂ₙ₊₁—(CH₂)ₘ}Boc.

28. The oligonucleotide reagent of claim 13, characterized by the following nominal formula (IV): 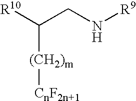
Wherein,
n is an integer from 4-12;
m is an integer from 1-4:
R⁹ is selected from the group consisting of H, Boc, Cbz, COCH₂CH₂CO2H, a fluorescent tag, a quencher tag, biotin, and a solid phase synthesis support; and
R¹⁰ is selected from the group consisting of CO₂H, CO₂CH₃, CO₂—(N-succinimidyl), CONH(CH₂)_{q}N-maleimide, CONH(CH₂)_{q}NHCOCH₂I, CONH(CH₂)_{q}NHCOCH₂Br, CONH(CH₂)_{q}OCH₂CH(OR⁸)CH₂OR⁷, CH₂OH, CH₂OP(R¹)OCH₂CH₂CN, CH₂OCH₂CH(OR⁸)CH₂OR⁷, CH₂OCH(CH₂OR⁷) CH₂OR⁸, CH₂O(CH₂)_{q}OR⁷, CH₂O(CH₂CH₂O)_{q}R⁷, and CH₂O(CH₂)_{q}—S—S—(CH₂)_{q}OR⁷, and in which group q is 2-12, R⁷ is one of H, COCH₂CH₂CO2H, DMTr, MMTr, a solid phase synthesis support, and P(R¹)OCH₂CH₂CN, R¹ is one of N(CH₃)₂, N(C₂H₅)₂, N(C₃H₇)₂, N(CH(CH₃)₂)₂, 1-pyrrolidinyl, 1-piperidinyl, 4-morpholinyl, and 1-imidazolyl, and R₈ is one of H, COCH₂CH₂CO2H, DMTr, MMTr, a solid phase synthesis support, and P(R¹)OCH₂CH₂CN, and when R⁷ and R⁸ are both present they are not identical.

29. The oligonucleotide reagent of claim 28, wherein said reagent comprises a fluorescent tag according to any of the following nominal compounds:

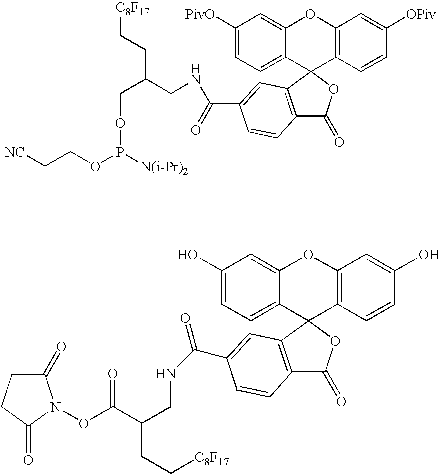

30. The oligonucleotide reagent of claim 28, wherein said reagent comprises a quencher tag according to any of the following nominal compounds:

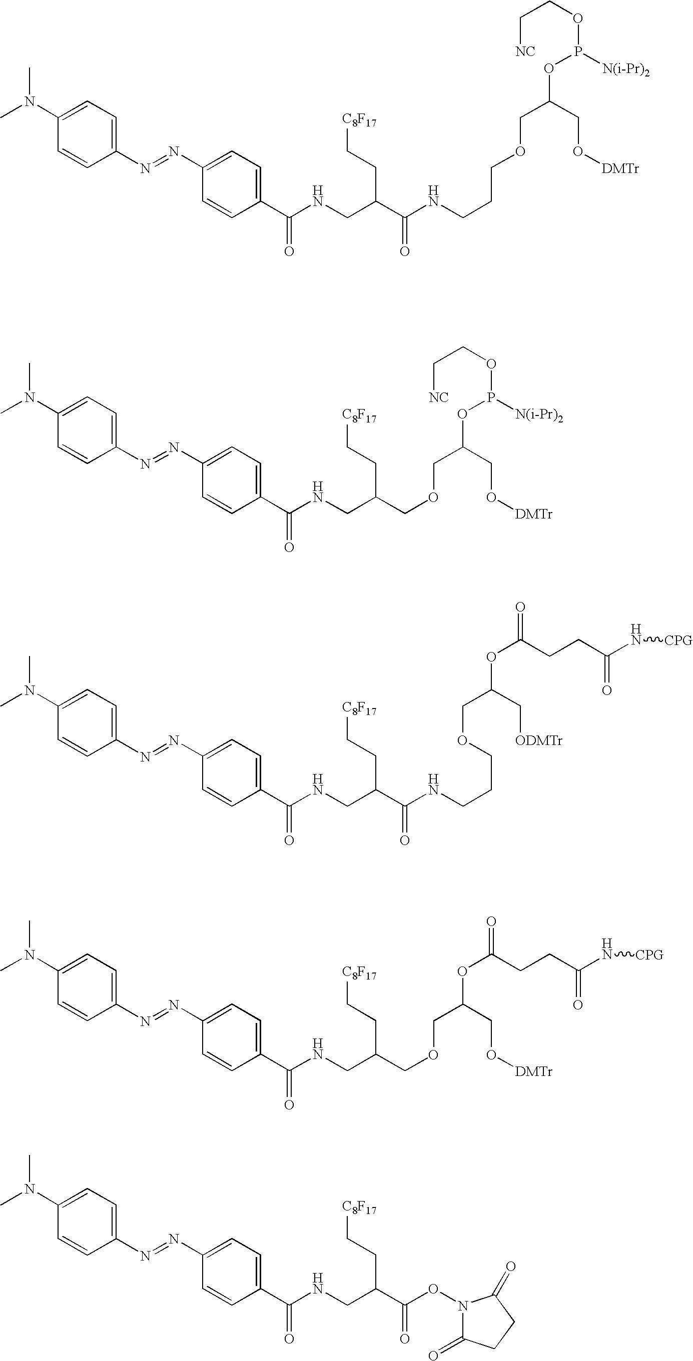

31. The oligonucleotide reagent of claim 13, characterized by the following nominal formula (V): 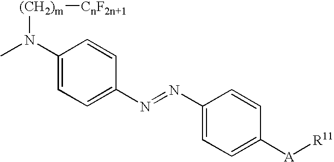
Wherein,
m is an integer from 1-4;
n is an integer from 4-12;
A is CO or SO₂; and
R¹¹ is selected from the group consisting of Cl, OH, OCH₃, O—(N-succinimidyl), NH(CH₂)ₜOCH₂CH(OR⁸)CH₂OR⁷, NH(CH₂)_{q}OR⁷, NH(CH₂)ₜO(CH₂CH₂O)_{q}R⁷, and NH(CH₂)_{q}—S—S—(CH₂)_{q}OR⁷ and in which group R⁷ is one of H, COCH₂CH₂CO2H, DMTr, MMTr, a solid phase synthesis support, and P(R¹)OCH₂CH₂CN, R¹ is one of N(CH₃)₂, N(C₂H₅)₂, N(C₃H₇)₂, N(CH(CH₃)₂)₂, 1-pyrrolidinyl, 1-piperidinyl, 4-morpholinyl, and 1-imidazolyl, and R₈ is one of H, COCH₂CH₂CO2H, DMTr, MMTr, a solid phase synthesis support, and P(R¹)OCH₂CH₂CN, and when R⁷ and R⁸ are both present they are not identical.

32. The oligonucleotide reagent of claim 31, wherein said reagent comprises a quencher tag according to any of the following nominal compounds: 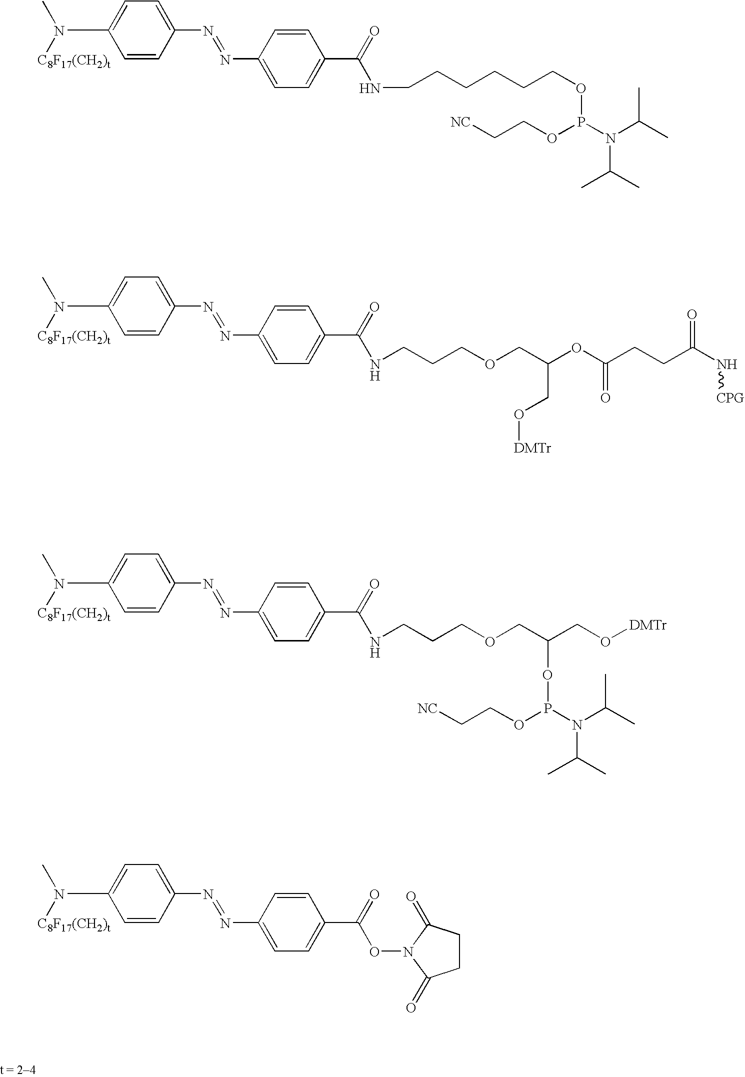
Wherein,
t is an integer from 2-4.
